(19) United States
(12) Patent Application Publication
(10) Pub. No.: US 2021/0003581 A1
(43) Pub. Date: Jan. 7, 2021

(54) IMMOBILIZED SUBSTRATE ENZYMATIC SURFACE ENHANCED RAMAN SPECTROSCOPY (SERS) ASSAYS

(71) Applicant: The Texas A&M University System, College Station, TX (US)

(72) Inventors: Gerard L. COTE, College Station, TX (US); Javier T. GARZA, College Station, TX (US)

(21) Appl. No.: 16/316,127

(22) PCT Filed: Aug. 7, 2017
(86) PCT No.: PCT/US2017/045700

§ 371 Date: Jan. 8, 2019

Related U.S. Application Data
(60) Provisional application No. 62/371,481, filed on Aug. 5, 2016.

(51) Int. Cl. G01N 33/58; G01N 33/543; G01N 21/65

(52) U.S. Cl. CPC G01N 33/581; G01N 21/658; G01N 33/543

(57) ABSTRACT

Provided are methods of quantifying analyte concentration in a sample by providing a pre-screened amount of an enzyme that has been pre-screened against the analyte in the sample; associating the pre-screened enzyme with a Raman reporter molecule that reacts with the enzyme to result in a change in the Raman spectrum pattern of the Raman reporter molecule; detecting the change in the Raman spectrum pattern of the Raman reporter molecule; and correlating the change (e.g., change rate) to the concentration of the analyte in the sample. Pre-screening may affect the concentration or activity of the enzyme in proportion to analyte concentration. Correlation may occur by correlating the change in the Raman spectrum pattern of the Raman reporter molecule to the concentration of the pre-screened amount of the enzyme, and correlating the concentration of the pre-screened amount of the enzyme to the concentration of the analyte in the sample.

### CROSS-REFERENCE TO RELATED APPLICATIONS

This application claims priority to U.S. Provisional Patent Application No. 62/371,481, filed on Aug. 5, 2016. The entirety of the aforementioned application is incorporated herein by reference.

### STATEMENT REGARDING FEDERALLY SPONSORED RESEARCH

Not applicable.

### BACKGROUND

Current analyte detection assays have limitations in terms of sensitivity and broad applicability. The present disclosure addresses the aforementioned limitations.

### SUMMARY

In some embodiments, the present disclosure pertains to methods of quantifying an analyte concentration in a sample by: (1) providing a pre-screened amount of an enzyme that has been pre-screened against the analyte in the sample; (2) associating the pre-screened enzyme with a Raman reporter molecule that reacts with the enzyme to result in a change in the Raman spectrum pattern of the Raman reporter molecule; (3) detecting the change in the Raman spectrum pattern of the Raman reporter molecule; and (4) correlating the change in the Raman spectrum pattern of the Raman reporter molecule to the concentration of the analyte in the sample.

In some embodiments, the pre-screening of the enzyme against the analyte in the sample affects the concentration of the enzyme or the activity of the enzyme in proportion to the amount of the analyte in the sample. In some embodiments, the enzyme is an enzyme-linked analyte. In some embodiments, the enzyme-linked analyte is pre-screened against the analyte by the following steps: (1) associating the analyte from the sample with an immobilized binding agent specific for the analyte, where the associating results in the binding of at least some of the analytes to the immobilized binding agent; (2) adding a known amount of the enzyme-linked analyte to the immobilized binding agent, where the adding results in the binding of at least some of the enzyme-linked analyte to the immobilized binding agent; and (3) separating the unbound enzyme-linked analyte to provide the pre-screened amount of the enzyme.

In some embodiments, the enzyme is an enzyme-linked analyte binding agent. In some embodiments, the enzyme-linked analyte binding agent is pre-screened against the analyte by the following steps: (1) associating the analyte from the sample with an immobilized binding agent specific for the analyte, where the associating results in the binding of at least some of the analytes to the immobilized binding agent; (2) adding a known amount of the enzyme-linked analyte binding agent to the immobilized binding agent, where the adding results in the binding of at least some of the enzyme-linked analyte binding agent to the analyte bound to the immobilized binding agent; and (3) separating the unbound enzyme-linked analyte binding agent to provide the pre-screened amount of the enzyme.

In further embodiments, the enzyme-linked analyte binding agent is pre-screened against the analyte by the following steps: (1) mixing a known amount of the enzyme-linked analyte binding agent with the analyte from the sample, where the mixing results in the binding of at least some of the enzyme-linked analyte binding agents to the analyte to form enzyme-linked analyte binding agent-analyte complexes; (2) associating the enzyme-linked analyte binding agent-analyte complexes with an immobilized binding agent specific for the analyte, where the associating results in the binding of at least some of the enzyme-linked analyte binding agent-analyte complexes to the immobilized binding agent; and (3) separating the unbound enzyme-linked analyte binding agents to provide the pre-screened amount of the enzyme.

In additional embodiments, the enzyme-linked analyte binding agent is pre-screened against the analyte by the following steps: (1) associating the analyte from the sample with a known amount of an enzyme-linked analyte binding agent, where the associating results in the binding of at least some of the analytes to the enzyme-linked analyte binding agent; (2) adding the enzyme-linked analyte binding agent to immobilized analytes, where the adding results in the binding of at least some of the enzyme-linked analyte binding agents to the immobilized analytes; and (3) separating the enzyme-linked analyte binding agents that are not bound to the immobilized analytes to provide the pre-screened amount of the enzyme.

In further embodiments, the enzyme-linked analyte binding agent is pre-screened against the analyte by the following steps: (1) associating a known amount of the enzyme-linked analyte binding agent with an immobilized binding agent specific for the enzyme-linked analyte binding agent, where the associating results in the binding of at least some of the enzyme-linked analyte binding agents to the immobilized binding agents; (2) adding the analyte from the sample to the immobilized binding agents, where the adding results in the binding of at least some of the enzyme-linked analyte binding agents to the analyte to form enzyme-linked analyte binding agent-analyte complexes, and where the binding facilitates the release of the enzyme-linked analyte binding agent-analyte complexes from the immobilized surface; and (3) separating the released enzyme-linked analyte binding agent-analyte complexes to provide the pre-screened amount of the enzyme.

In some embodiments, the enzyme is in the form of a free enzyme. In some embodiments, the free enzyme is pre-screened against the analyte in the presence of an enzyme inhibitor. In some embodiments, the enzyme is pre-screened by the following steps: (1) associating a known amount of the enzyme with an enzyme inhibitor that includes an analyte binding agent, where the associating results in the binding of at least some of the enzyme inhibitors to at least some of the enzymes to form enzyme-inhibitor complexes; and (2) adding the analyte from the sample to the enzyme-inhibitor complexes. The adding results in the binding of at least some of the analytes to the analyte binding agent of the enzyme-inhibitor complexes. Thereafter, the binding counteracts the inhibition of at least some of the enzymes by the enzyme inhibitors. As a result, the enzymes that are not inhibited by the enzyme inhibitors provide the pre-screened amount of the enzyme.

In some embodiments, the enzyme is pre-screened by associating the analyte from the sample with a known amount of the enzyme and an enzyme inhibitor that includes an analyte binding agent. The association results in the binding of at least some of the analytes to the analyte binding agent of the enzyme inhibitor. Thereafter, the binding facilitates the inhibition of at least some of the enzymes by the enzyme inhibitor. As a result, the enzymes that are not inhibited provide the pre-screened amount of the enzyme.

The reaction of a pre-screened enzyme with a Raman reporter molecule can result in a change in the Raman spectrum pattern of the Raman reporter molecule by various mechanisms. For instance, in some embodiments, the reaction of the enzyme with the Raman reporter molecule results in a structural change in the Raman reporter molecule.

The change in the Raman spectrum pattern of the Raman reporter molecule can be detected in various manners. For instance, in some embodiments, the change in the Raman spectrum pattern of the Raman reporter molecule is detected over time (e.g., real-time). In some embodiments, the detected change in the Raman spectrum pattern of the Raman reporter molecule is proportional to the amount of the pre-screened enzyme. In some embodiments, the detected change includes a detected change rate. In some embodiments, the detected change in the Raman spectrum pattern of the Raman reporter molecule is correlated to the concentration of the analyte in the sample by: (1) correlating the change (e.g., change over time) in the Raman spectrum pattern of the Raman reporter molecule to the concentration of the pre-screened amount of the enzyme; and (2) correlating the concentration of the pre-screened amount of the enzyme to the concentration of the analyte in the sample. In some embodiments, the correlating includes comparing the change (e.g., change over time) in the Raman spectrum pattern of the Raman reporter molecule to a calibration curve that represents different known amounts of the analyte.

### FIGURES

FIG. 1 provides schemes of methods for quantifying an analyte concentration in a sample (FIG. 1A) and pre-screening enzymes against analytes (FIGS. 1B-1G).

FIG. 2 provides a scheme of a competitive binding sequential saturation assay embodiment for quantifying an analyte concentration in a sample.

FIG. 3 provides a scheme of a sandwich assay embodiment for quantifying an analyte concentration in a sample.

FIG. 4 provides a scheme of a direct assay embodiment for quantifying an analyte concentration in a sample.

FIG. 5 provides a scheme of an enzymatic reaction of tyrosine with tyrosinase.

FIG. 6 provides various surface enhanced Raman spectroscopy (SERS) spectra.

FIG. 7 provides spectra of various monitored ratios over time.

FIG. 8 provides a scheme of an enzymatic reaction with highlighted Raman peaks assignments.

FIG. 9 provides an SERS Spectrum of L-Tyrosine, tyrosinase, product, and melanin.

FIG. 10 shows the SERS spectra of the reaction components at different steps of the surface reaction.

FIG. 11 illustrates various strategies for enzyme and protein conjugation.

FIG. 12 shows data of responses caused by different concentrations of enzymes conjugated with cardiac Troponin I (cTnI).

FIG. 13 shows an enzyme-linked molecule and an antibody conjugated on turbobeads.

FIG. 14 shows the SERS spectrum response of enzyme-troponin I and antibody-turbobeads.

FIG. 15 shows a reproducibility test.

FIG. 16 provides a scheme of an enzyme-linked molecule release assay embodiment for quantifying an analyte concentration in a sample.

FIG. 17 provides a scheme of a directly proportional inhibiting assay embodiment for quantifying an analyte concentration in a sample.

FIG. 18 provides a scheme of an inversely proportional inhibiting assay embodiment for quantifying an analyte concentration in a sample.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Analytical biochemical assays are commonly used by many industries to measure the presence and concentration of a substance (e.g., a chemical compound or biomolecule). This information can then be used as diagnostic, sensing, or monitoring tools.

Common analytical biochemical assays include immunoassays, such as the enzyme-linked immunosorbent assay (ELISA). ELISA uses an enzyme attached to an antibody that interacts with the antigen of interest or an antibody attached to the antigen of interest. After some time, unbound complexes are washed. Thereafter, a substrate is added that reacts with the enzyme to produce a change in absorbance, fluorescence intensity, or surface enhanced Raman spectroscopy (SERS) intensity.

Other assays use fluorophore labeled antibodies to monitor the signal intensity without the need of enzymes. However, very few assays that monitor the change in peaks of a SERS spectrum have been reported.

Furthermore, the SERS-based assays that have been reported are usually very specific. For example, in one reported assay to monitor pH, the Raman active molecule is protonated or deprotonated as the pH changes, modifying its SERS spectrum. In another reported assay, the Raman active molecule spectrum changes as glucose binds to the molecule to measure glucose levels.

As such, a need exists for the development of more broadly applicable SERS-based assays for detecting the concentration of various analytes. Various aspects of the present disclosure address the aforementioned need.

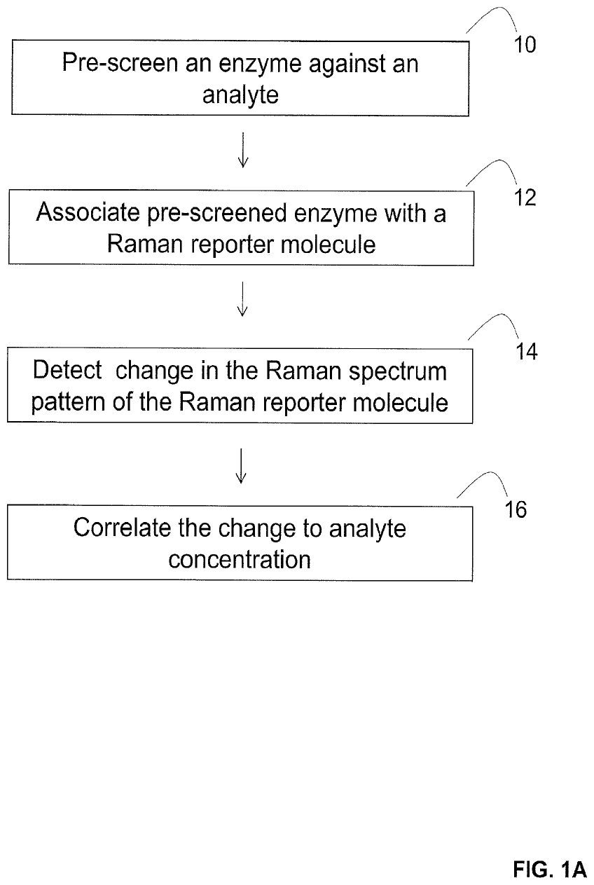

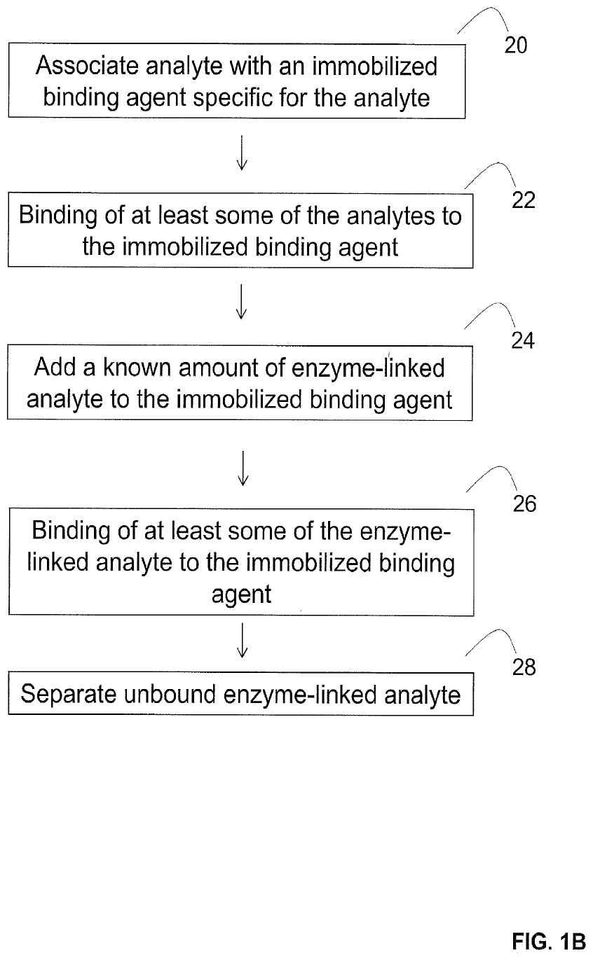

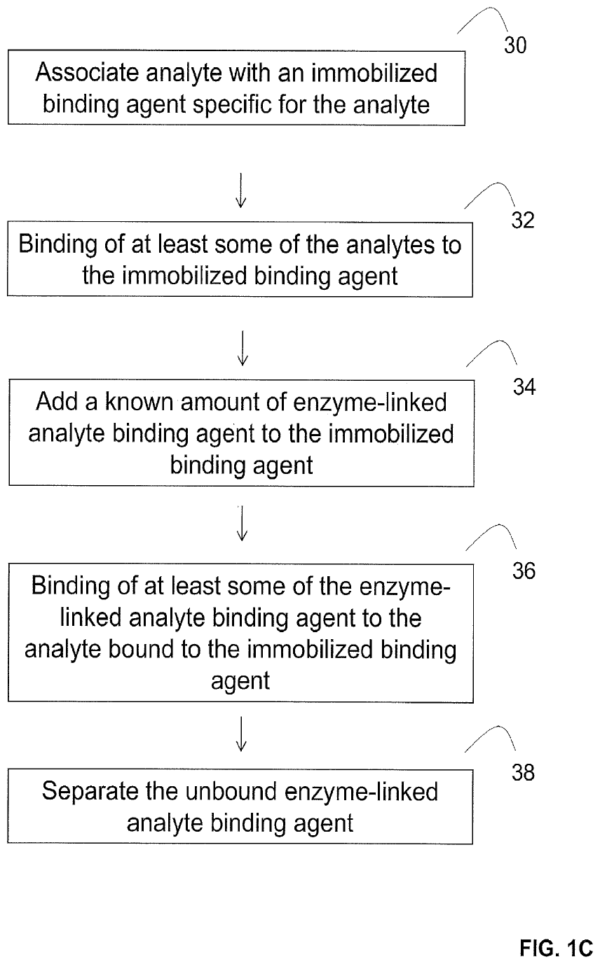

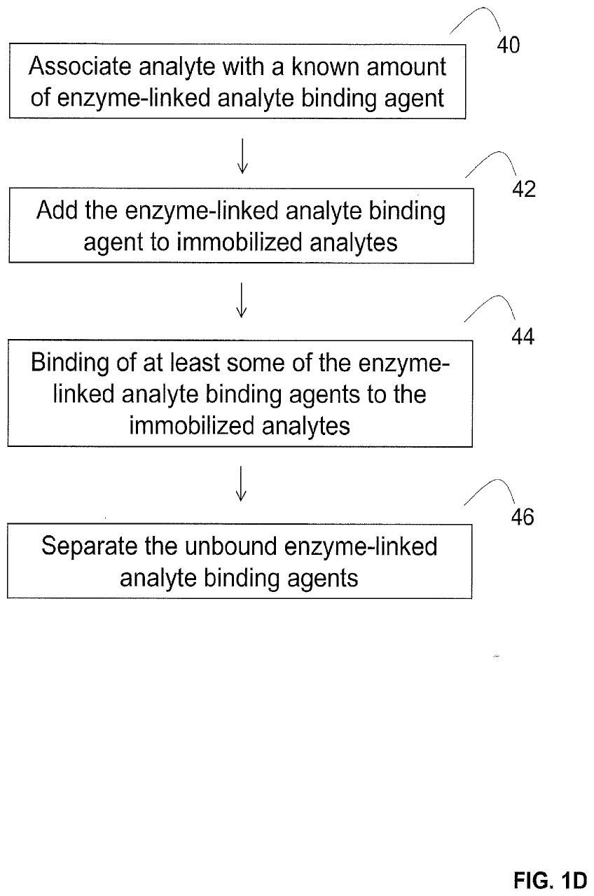

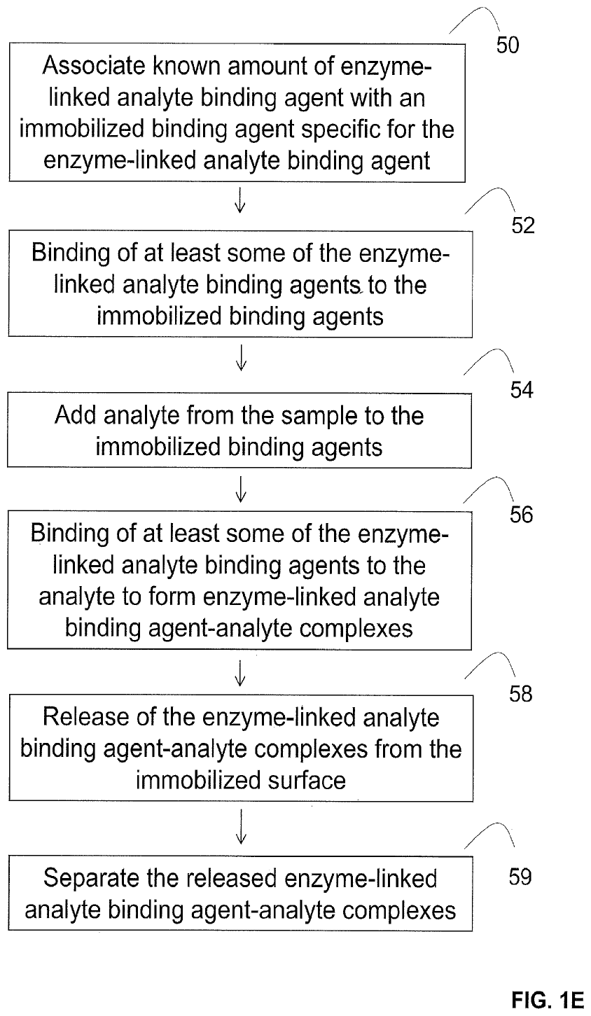

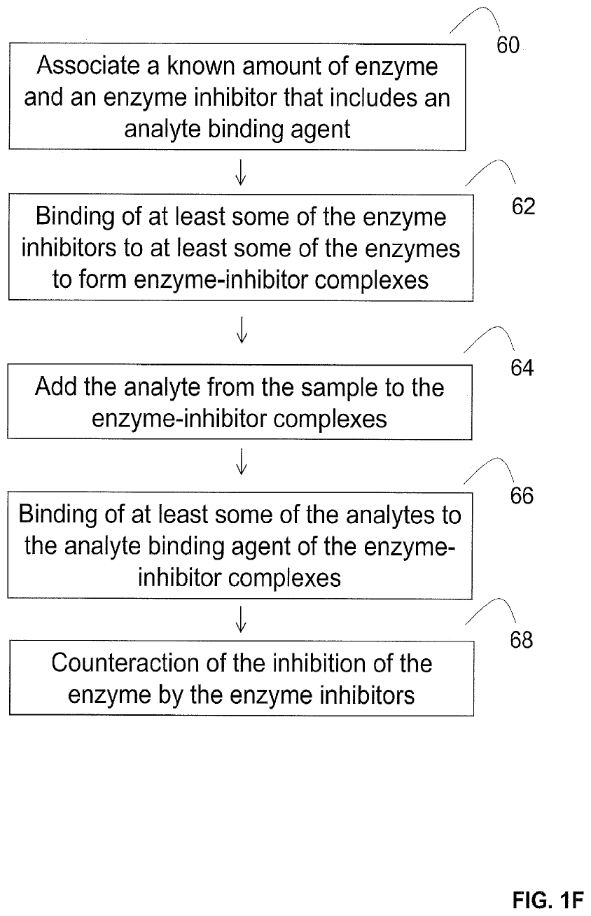

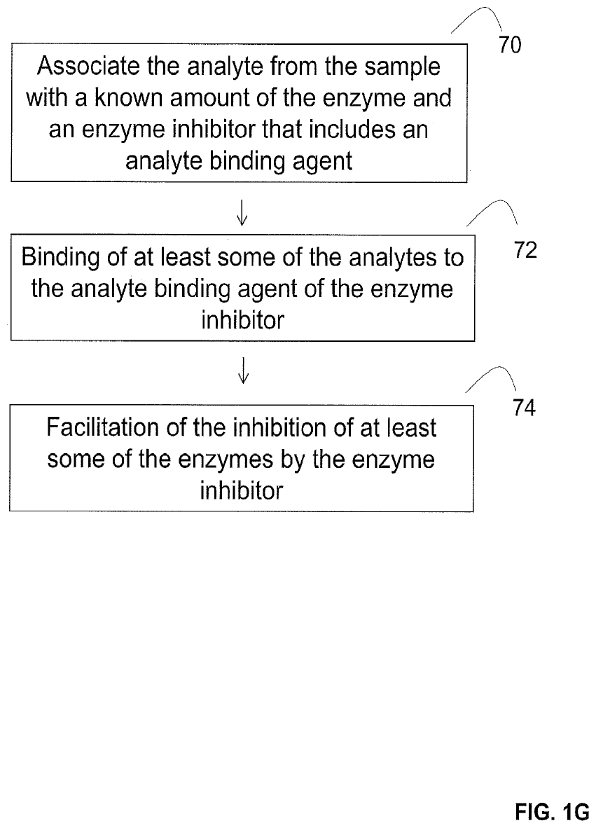

In some embodiments, the present disclosure provides methods of quantifying an analyte concentration in a sample. In some embodiments illustrated in FIG. 1A, the methods of the present disclosure include: providing an amount of an enzyme that has been pre-screened against the analyte in the sample (step 10); associating the pre-screened enzyme with a Raman reporter molecule such that the Raman reporter molecule reacts with the enzyme to result in a change in the Raman spectrum pattern of the Raman reporter molecule (step 12); detecting the change in the Raman spectrum pattern of the Raman reporter molecule (step 14); and correlating the change in the Raman spectrum pattern of the Raman reporter molecule to the concentration of the analyte in the sample (step 16).

As set forth in more detail herein, the methods of the present disclosure can have various embodiments. In particular, various types of enzymes may be pre-screened against various analytes in various manners. Moreover, various methods may be utilized to detect changes in the Raman spectrum patterns of various Raman reporter molecules and correlate those changes to the concentration of the analyte in the sample.

Enzymes

The methods of the present disclosure can utilize various types of enzymes. In some embodiments, the enzyme is in the form of a free enzyme that is not linked to other molecules. In some embodiments, the enzyme may be linked to other molecules by various bonds, such as covalent bonds. For instance, in some embodiments, the enzyme is an enzyme-linked analyte. In some embodiments, the enzyme is an enzyme-linked analyte binding agent.

The enzymes of the present disclosure can also have various enzyme components that react with Raman reporter molecules. For instance, in some embodiments, the enzyme component of the enzyme is tyrosinase. The use of other enzymes that are capable of reacting with Raman reporter molecules can also be envisioned.

Pre-Screening of Enzymes Against Analytes

The enzymes of the present disclosure can be pre-screened against an analyte in various manners. In addition, the pre-screening of the enzyme against the analyte in the sample can have various effects on the enzyme. For instance, in some embodiments, the pre-screening of the enzyme against the analyte in the sample affects the concentration of the enzyme in proportion to the amount of the analyte in the sample. In some embodiments, the pre-screening of the enzyme against the analyte in the sample reduces the concentration of the enzyme in proportion to the amount of the analyte in the sample. In some embodiments, the pre-screening of the enzyme against the analyte in the sample increases the concentration of the enzyme in proportion to the amount of the analyte in the sample.

In some embodiments, the pre-screening of the enzyme against the analyte in the sample affects the enzymatic activity of the enzyme in proportion to the amount of the analyte in the sample. For instance, in some embodiments, the pre-screening of the enzyme against the analyte in the sample reduces the enzymatic activity of the enzyme in proportion to the amount of the analyte in the sample. In some embodiments, the pre-screening of the enzyme against the analyte in the sample increases the enzymatic activity of the enzyme in proportion to the amount of the analyte in the sample. In some embodiments, the pre-screening of the enzyme against the analyte in the sample reduces the concentration and enzymatic activity of the enzyme in proportion to the amount of the analyte in the sample.

Exemplary methods that may be utilized to provide pre-screened enzymes are illustrated in FIGS. 1B-1G. For instance, in some embodiments illustrated in FIG. 1B where the enzyme is an enzyme-linked analyte, the enzyme-linked analyte can be pre-screened against the analyte by associating the analyte from the sample with an immobilized binding agent specific for the analyte (step 20) to result in the binding of at least some of the analytes to the immobilized binding agent (step 22). Next, a known amount of the enzyme-linked analyte is added to the immobilized binding agent (step 24) to result in the binding of at least some of the enzyme-linked analyte to the immobilized binding agent (step 26). Thereafter, the unbound enzyme-linked analyte is separated to provide the pre-screened amount of the enzyme (step 28).

In other embodiments illustrated in FIG. 1C where the enzyme is an enzyme-linked analyte binding agent, the enzyme-linked analyte binding agent can be pre-screened against the analyte by associating the analyte from the sample with an immobilized binding agent specific for the analyte (step 30). The associating results in the binding of at least some of the analytes to the immobilized binding agent (step 32). Thereafter, a known amount of the enzyme-linked analyte binding agent is added to the immobilized binding agent (step 34) to result in the binding of at least some of the enzyme-linked analyte binding agent to the analyte bound to the immobilized binding agent (step 36). Next, the unbound enzyme-linked analyte binding agent is separated to provide the pre-screened amount of the enzyme (step 38).

In another embodiment, the enzyme-linked analyte binding agent can be mixed with the analyte from the sample before adding them to the immobilized binding agent. In such embodiments, the pre-screening methods of the present disclosure can involve mixing a known amount of an enzyme-linked analyte binding agent with an analyte from the sample to result in the binding of at least some of the enzyme-linked analyte binding agents to the analyte to form enzyme-linked analyte binding agent-analyte complexes. Thereafter, the enzyme-linked analyte binding agent-analyte complexes are associated with an immobilized binding agent specific for the analyte. The associating results in the binding of at least some of the enzyme-linked analyte binding agent-analyte complexes to the immobilized binding agent. Next, the unbound enzyme-linked analyte binding agents are separated to provide the pre-screened amount of the enzyme.

In additional embodiments illustrated in FIG. 1D, the enzyme-linked analyte binding agent can be pre-screened against the analyte by associating the analyte from the sample with a known amount of enzyme-linked analyte binding agent to result in the binding of at least some of the analytes to the enzyme-linked analyte binding agent (step 40). Thereafter, the enzyme-linked analyte binding agent is added to immobilized analytes (step 42) to result in the binding of at least some of the enzyme-linked analyte binding agents to the immobilized analytes (step 44). Next, the enzyme-linked analyte binding agents that are not bound to the immobilized analytes are separated to provide the pre-screened amount of the enzyme (step 46).

In further embodiments illustrated in FIG. 1E, the enzyme-linked analyte binding agent is pre-screened against the analyte by associating a known amount of the enzyme-linked analyte binding agent with an immobilized binding agent specific for the enzyme-linked analyte binding agent (step 50) such that the associating results in the binding of at least some of the enzyme-linked analyte binding agents to the immobilized binding agents (step 52). Next, the analyte from the sample is added to the immobilized binding agents (step 54) such that the adding results in the binding of at least some of the enzyme-linked analyte binding agents to the analyte to form enzyme-linked analyte binding agent-analyte complexes (step 56). This in turn results in the facilitation of the release of the enzyme-linked analyte binding agent-analyte complexes from the immobilized surface (step 58). Thereafter, the released enzyme-linked analyte binding agent-analyte complexes are separated to provide the pre-screened amount of the enzyme (step 59).

In some embodiments (e.g., embodiments where the enzyme is in the form of a free enzyme), the enzyme can be pre-screened against the analyte in the presence of an enzyme inhibitor. For instance, in some embodiments illustrated in FIG. 1F, the enzyme is pre-screened by associating a known amount of the enzyme with an enzyme inhibitor that includes an analyte binding agent (step 60) such that the association results in the binding of at least some of the enzyme inhibitors to at least some of the enzymes to form enzyme-inhibitor complexes (step 62). In some embodiments, the enzyme inhibitors that do not bind to the enzymes are washed away. Thereafter, the analyte from the sample is added to the enzyme-inhibitor complexes (step 64). The adding results in the binding of at least some of the analytes to the analyte binding agent of the enzyme-inhibitor complexes (step 66).

Thereafter, the binding of at least some of the analytes to the analyte binding agent of the enzyme-inhibitor complexes counteracts the inhibition of at least some of the enzymes by the enzyme inhibitors (step 68). As a result, the enzymes that are not inhibited by the enzyme inhibitors provide the pre-screened amount of the enzyme.

The binding of at least some of the analytes to the analyte binding agent of the enzyme-inhibitor complexes can counteract the inhibition of at least some of the enzymes by the enzyme inhibitors in various manners. For instance, in some embodiments, the binding counteracts the inhibition of at least some of the enzymes by the enzyme inhibitors by facilitating the release of at least some of the enzyme inhibitors from at least some of the enzyme-inhibitor complexes in the sample.

In other embodiments, the binding counteracts the inhibition of at least some of the enzymes by the enzyme inhibitors by inactivating or modifying the enzyme inhibition activity of the enzyme inhibitor. For instance, in some embodiments, the binding inactivates or modifies the enzyme inhibition activity of the enzyme inhibitors by modifying the structure of the enzyme inhibitors. In some embodiments, the structure of the enzyme inhibitors is modified by disassembling the enzyme inhibitor (e.g., breaking the inhibitor while parts of the inhibitor are still bound to the enzyme).

In additional embodiments illustrated in FIG. 1G, an enzyme is pre-screened against the analyte by associating the analyte from the sample with a known amount of the enzyme and an enzyme inhibitor that includes an analyte binding agent (step 70). The association results in the binding of at least some of the analytes to the analyte binding agent of the enzyme inhibitor (step 72). Thereafter, the binding facilitates the inhibition of at least some of the enzymes by the enzyme inhibitor (step 74). As a result, the enzymes that are not inhibited provide the pre-screened amount of the enzyme.

The binding of at least some of the analytes to the analyte binding agent of the enzyme inhibitor can facilitate the inhibition of at least some of the enzymes by the enzyme inhibitor in various manners. For instance, in some embodiments, the binding facilitates the inhibition of at least some of the enzymes by the enzyme inhibitor by facilitating the binding of the enzyme inhibitor to at least some of the enzymes in the sample. In some embodiments, the binding facilitates the inhibition of at least some of the enzymes by the enzyme inhibitor by activating the inhibitor. In some embodiments, the activation occurs by modifying the structure of the enzyme inhibitor (e.g., by joining or assembling parts of the inhibitor) such that the modified inhibitor inhibits the enzyme.

Handling of Pre-Screened Enzymes

Pre-screened enzymes can be handled in different manners. For instance, in some embodiments, the present disclosure can utilize various methods to separate unbound enzymes from a sample to provide pre-screened enzymes. In some embodiments, the separation occurs by decanting, centrifugation, fluid flow in channels, magnetic separation, and combinations of such steps. In some embodiments (e.g., embodiments where the enzyme is inhibited by an enzyme inhibitor), no separation is necessary. Additional separation methods can also be envisioned.

Various methods may also be utilized to associate pre-screened enzymes with a Raman reporter molecule. For instance, in some embodiments, the association occurs by mixing. In some embodiments, the Raman reporter molecule is a substrate to the enzyme in the presence of oxygen. As such, in some embodiments, oxygen is also added.

Raman Reporter Molecules

The methods of the present disclosure can utilize various Raman reporter molecules. Raman reporter molecules generally refer to molecules that have a Raman spectrum. In some embodiments of the present disclosure, the Raman reporter molecules are also substrates for enzymes. For instance, in some embodiments where the enzyme contains tyrosinase, the Raman reporter molecule is tyrosine. The use of additional combinations of enzymes and Raman reporter molecules can also be envisioned.

The Raman reporter molecules of the present disclosure can be in various forms. For instance, in some embodiments, the Raman reporter molecule is immobilized on a surface. In some embodiments, the Raman reporter molecule is immobilized on a surface by adsorption to the surface. In some embodiments, the Raman reporter molecule is immobilized on a surface by covalent attachment to the surface. In some embodiments, the immobilization of the Raman reporter molecule on a surface allows more effective monitoring of the change in the Raman spectrum pattern of the Raman reporter molecule (e.g., monitoring a change before and after adding an enzyme). In some embodiments, the immobilization of the Raman reporter molecule on a surface can also prevent other molecules that might have a Raman spectrum from attaching or adsorbing to a surface and affecting the Raman spectrum (e.g., creating noise or affecting the Raman spectrum in any way).

In some embodiments, the surface is a surface enhanced Raman spectroscopy (SERS) active area. In some embodiments, the surface is in the form of a metallic layer. In some embodiments, the surface is in the form of nanoparticles. In some embodiments, the surface is in the form of gold or silver metallic layers. In some embodiments, the surface is in the form of gold or silver nanoparticles.

Change in the Raman Spectrum Pattern of the Raman Reporter Molecules

The association of an enzyme with a Raman reporter molecule can have various effects on the Raman reporter molecule. For instance, in some embodiments, the reaction of the Raman reporter molecule with the enzyme results in a structural change in the Raman reporter molecule. In some embodiments, the enzyme eliminates or adds atoms within the Raman reporter molecule. In some embodiments, the enzyme changes the bonds within the Raman reporter molecule.

The enzymes of the present disclosure can change the Raman spectrum pattern of the Raman reporter molecules of the present disclosure in various manners. For instance, in some embodiments, the enzymes of the present disclosure cause changes in the Raman spectrum peaks of the Raman reporter molecules, such as causing the peaks to appear, disappear, broaden, narrow, or change height. In some embodiments, the change can be proportional to the amount of the pre-screened amount of the enzyme. In some embodiments, the Raman reporter molecule is modified in a rate dependent manner based on the specific enzymatic reaction rate and the amount of pre-screened enzyme present.

Detecting the Change in the Raman Spectrum Pattern of the Raman Reporter Molecules

The change in the Raman spectrum pattern of the Raman reporter molecule can also be detected in various manners. For instance, in some embodiments, the change is detected over time. In some embodiments, the change is detected in real-time. In some embodiments, the change is detected before, during and after associating the enzyme with the Raman reporter molecule.

Moreover, the change in the Raman spectrum pattern of the Raman reporter molecule can be represented in various manners. For instance, in some embodiments, the change in the Raman spectrum pattern of the Raman reporter molecule can be represented by changes in at least one of peak positions, peak intensities, peak ratios, areas under the peaks, and combinations of such changes. In some embodiments, the detected change includes a detected change rate.

Correlation to Analyte Concentration

Various methods may also be utilized to correlate a change (e.g., a change over time) in the Raman spectrum pattern of a Raman reporter molecule to the concentration of an analyte in a sample. For instance, in some embodiments, the correlation occurs by correlating the change (e.g., a change over time) in the Raman spectrum pattern of the Raman reporter molecule to the concentration of the pre-screened amount of the enzyme, and then correlating the concentration of the pre-screened amount of the enzyme to the concentration of the analyte in the sample.

In some embodiments, the correlation occurs by comparing the change (e.g., a change over time) in the Raman spectrum pattern of the Raman reporter molecule to a calibration curve that represents different known amounts of the analyte. In some embodiments, the calibration curve correlates the changes in a Raman spectrum pattern at a specific point in time to the Raman spectrum patterns of different known amounts of the analyte to determine the unknown concentrations of the analyte.

In some embodiments, low concentrations of enzymes produce a slower change in the Raman spectrum compared to high concentrations of enzymes. In some embodiments, certain peak ratios are monitored after a specific amount of time has elapsed after the enzymes are introduced to the Raman reporter molecules. The peak values at the specified time are then correlated to the corresponding concentrations of enzymes present to create the calibration curve. The concentrations of enzymes are then correlated to their corresponding amounts of analyte present.

The correlation of a change in the Raman spectrum pattern of a Raman reporter molecule to the concentration of an analyte in a sample can be performed for various periods of time. For instance, in some embodiments, low and high concentrations of enzymes reacting with a Raman reporter molecule may cause the same change in the Raman spectrum pattern after a long period of time (e.g., 1-2 hours). However, the change in the Raman spectrum pattern may be more distinguishable during a shorter period of time (e.g., 1-30 minutes).

Analytes

The methods of the present disclosure can be utilized to detect the concentration of various analytes. For instance, in some embodiments, the analytes of the present disclosure include, without limitation, antigens, proteins, peptides, small molecules, DNA strands, oligonucleotides, and combinations thereof. In some embodiments, the analytes of the present disclosure include antigens, such as cardiac Troponin I.

The analytes of the present disclosure can be in a sample in various forms. For instance, in some embodiments, the analytes of the present disclosure are in the form of a solution in a sample.

Binding Agents

The methods of the present disclosure can utilize various types of binding agents (e.g., analyte binding agents or binding agents specific for enzyme-linked analyte binding agents). For instance, in some embodiments, the binding agents include, without limitation, antibodies, aptamers, haptens, DNA strands, oligonucleotides, and combinations thereof. In some embodiments, the binding agents include specific DNA strands that bind to a corresponding DNA strand. In some embodiments, the binding agents include antibodies, such as antibodies specific for cardiac Troponin I. In some embodiments, the binding agents include aptamers.

Immobilization

Various components of the present disclosure (e.g., analyte binding agents, analytes, binding agents specific for enzyme-linked analyte binding agents) can be immobilized in various manners. For instance, in some embodiments, the one or more components can be immobilized on a solid support. In some embodiments, the one or more components can be immobilized on particles. In some embodiments, the one or more components can be immobilized on beads. In some embodiments, the one or more components can be immobilized within microfluidic channels, test strips, wells, or combinations of such structures.

Applications and Advantages

The methods of the present disclosure provide flexible platforms that are not constrained to specific applications. Furthermore, the methods of the present disclosure have the potential of making highly sensitive and precise measurements that are robust. Furthermore, the methods of the present disclosure have a built-in calibration component since they induce a conformational change to a Raman reporter molecule by an enzyme that transforms the spectrum and not just an overall intensity change in the spectrum.

In some embodiments, the methods of the present disclosure can also be utilized for the simultaneous monitoring of different analytes. For instance, in some embodiments where peaks in surface enhanced Raman spectroscopy are narrow, different analytes can be monitored simultaneously by using different enzyme-Raman reporter molecule pairings. In some embodiments, the multiplexing capability can be rationally designed so that the spectral changes from different pairings do not overlap and can be distinguished in the same measurement. In some embodiments, the different enzymes and Raman reporter molecules can be designed so that they do not interact with each other when they are mixed in the same solution.

Accordingly, the methods of the present disclosure can be utilized to monitor the concentration of many different analytes in numerous industries that utilize analytical biochemical assays. Such industries can include, without limitation, the food sciences industries, the biomedical industries (e.g., forensics, veterinary and pharmaceutical industries), and the environmental industries. The methods of the present disclosure can also be utilized to monitor analytes for numerous applications, such as quality control, drug analysis (e.g., in the pharmaceutical industry to develop and analyze drugs), forensics (e.g., to obtain information from biological samples), life science research (e.g., to study biochemical phenomena), and in the medical field (e.g., to diagnose conditions and make treatment decisions).

### Additional Embodiments

Reference will now be made to more specific embodiments of the present disclosure and experimental results that provide support for such embodiments. However, Applicants note that the disclosure herein is for illustrative purposes only and is not intended to limit the scope of the claimed subject matter in any way.

### EXAMPLE 1

### Enzymatic Assays

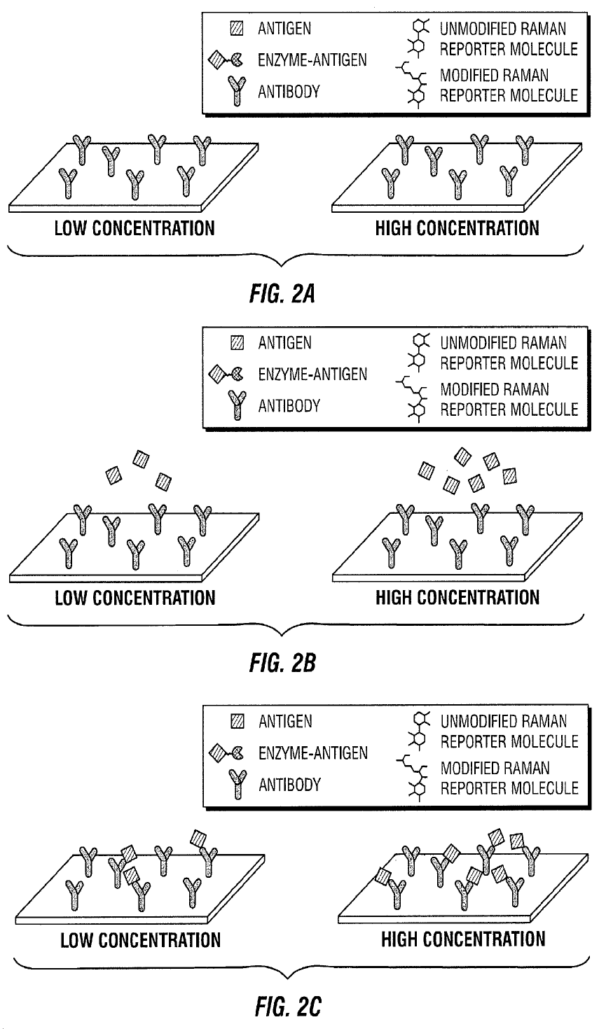

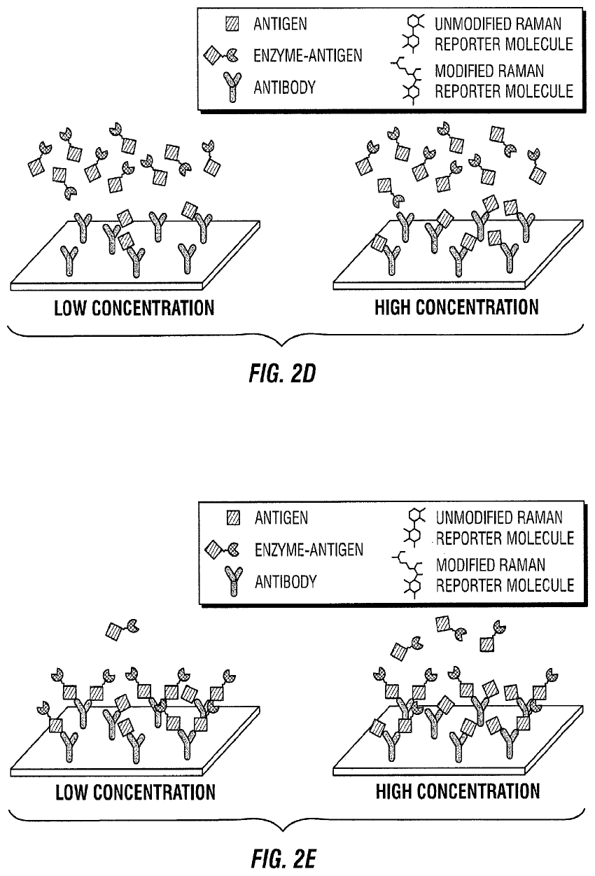

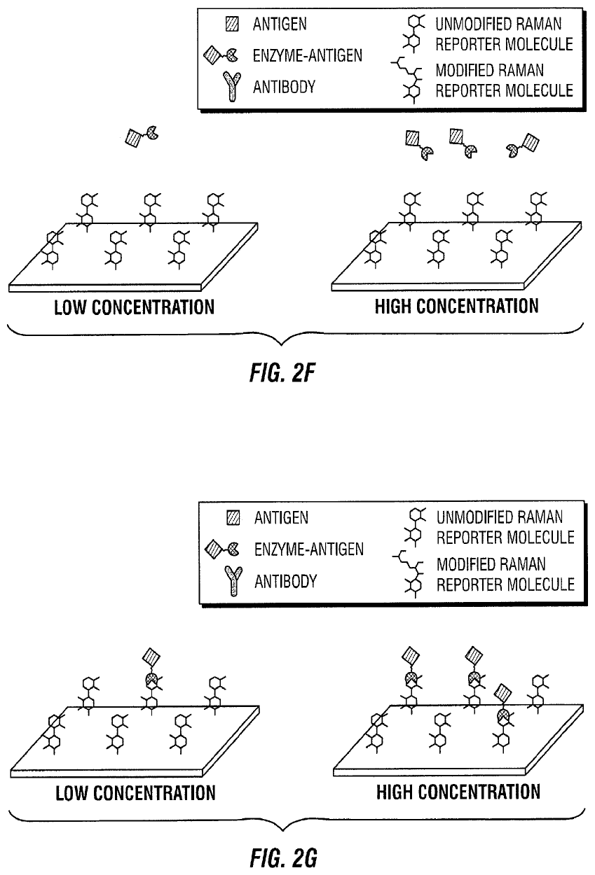

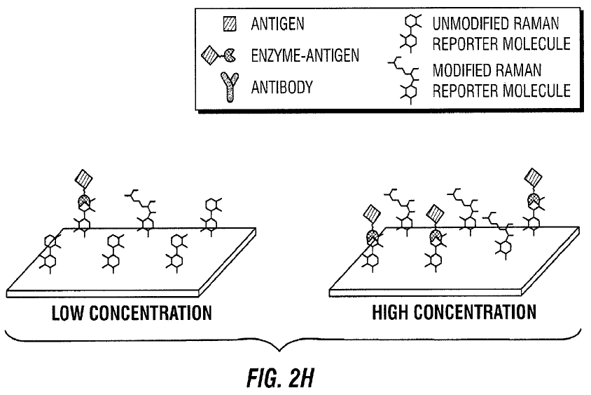

This Example provides three different embodiments of enzymatic assays. Such examples are illustrated in FIGS. 2-4.

FIG. 2 illustrates a competitive binding sequential saturation assay embodiment. In this embodiment, antibodies are first immobilized on a solid support (FIG. 2A). Thereafter, the sample to be measured that contains the antigen of interest is added to the area with immobilized antibodies (FIG. 2B). As a result, the antigen in the sample of interest binds to its antibody (FIG. 2C). After some time, a known amount of enzyme-antigen complexes are introduced to the area with immobilized antibodies and sample of interest (FIG. 2D). The enzyme-antigen complexes then bind to the free antibody binding sites, which are proportional to the amount of antigen present in the sample of interest. (FIG. 2E). Next, the free enzyme-antigen complexes that did not bind to the antibody because it was occupied are transported to a surface enhanced Raman spectroscopy (SERS) active area with Raman active molecules immobilized on the SERS substrate (FIG. 2F). This SERS active area consists of a metallic layer, nanoparticles, or any other SERS substrate with Raman active compounds attached. Thereafter, the enzyme-antigen complexes react with their immobilized Raman active substrate (FIG. 2G). As a result of the reaction, the Raman active molecule is changed by the enzyme (FIG. 2H). This change is quantified in time as the Raman spectrum changes (FIG. 2I).

In particular, the Raman spectrum of the known Raman active compound immobilized on the SERS substrate is monitored as it changes due to the enzymatic modification caused by the enzyme-antigen that reacts with it. The chemical changes in the Raman active molecule cause a change in the SERS spectral lines that are monitored. At specific points in time, the change of the SERS spectrum is proportional to the amount of enzyme-biomolecule present, which in turn is proportional to the amount of the antigen of interest present. Therefore, the concentration of the antigen can be determined.

The Raman spectrum changes in time proportionally to the amount of enzyme-antigen present in the SERS active area. For instance, in the low concentration cases (left panel of FIGS. 2A-I), it takes about 10 minutes for the red spectrum to change to the pink spectrum (FIG. 2I left panel, pink line), where it only takes 5 minutes to achieve the same change in the high concentration cases (FIG. 2I right panel, blue line). After 10 minutes, the spectrum of the high concentration case (FIG. 2I, right panel, pink line) has considerably changed compared to the spectrum at 0 minutes (FIG. 2I, right panel, red line) and the spectrum of the low concentration case at 10 minutes (FIG. 2I, left panel, pink line).

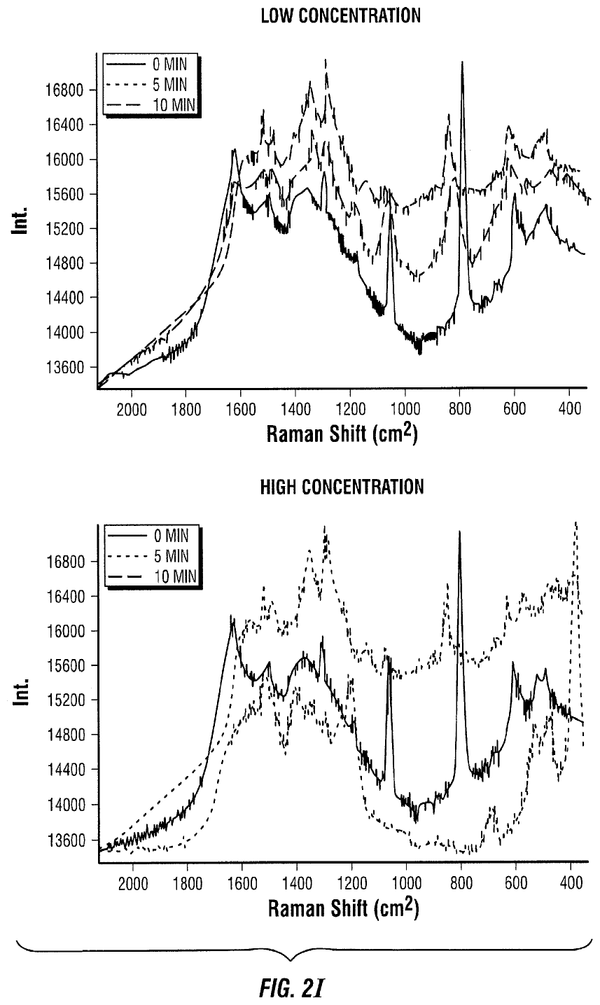

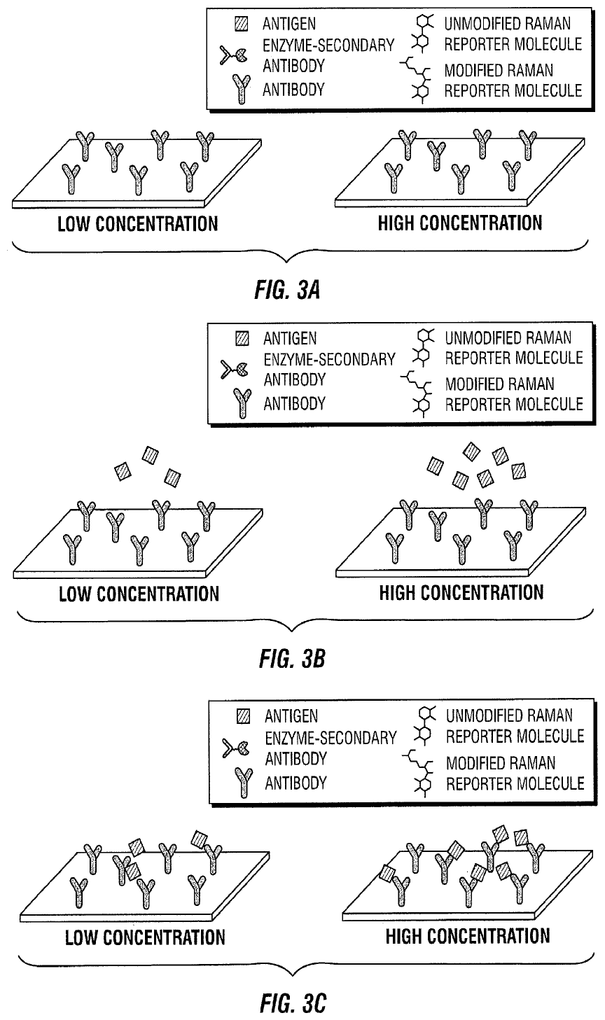

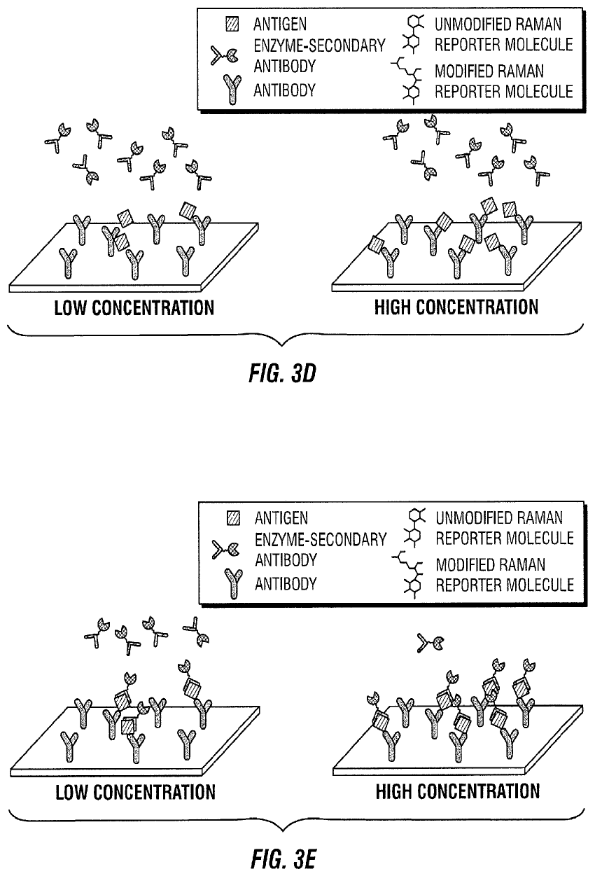

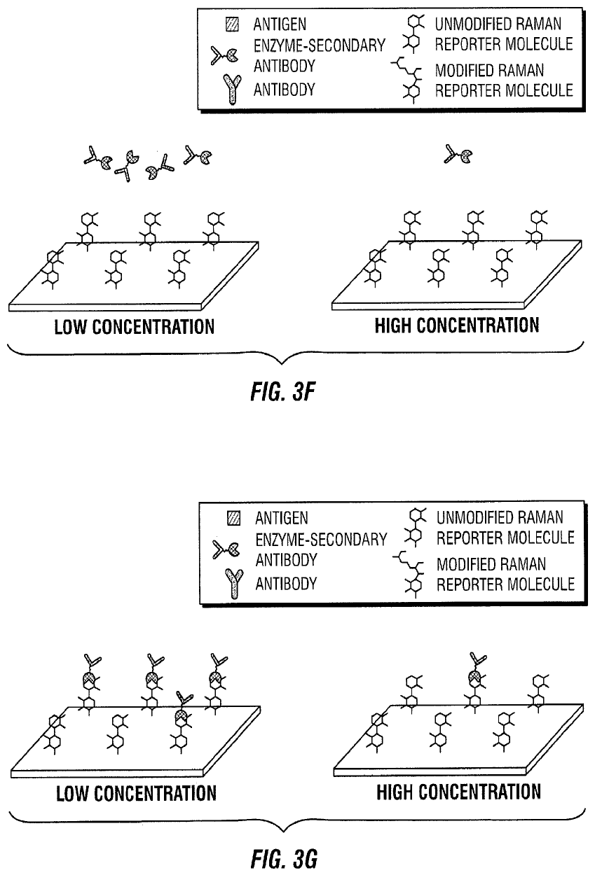

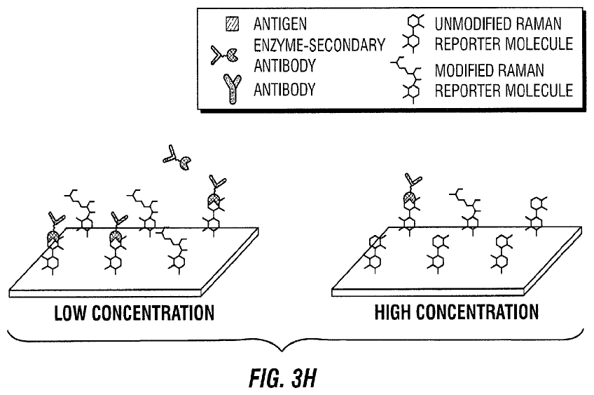

FIG. 3 illustrates a sandwich assay embodiment where antibodies are immobilized on a solid support (FIG. 3A). The sample to be measured is added to the area with immobilized antibodies (FIG. 3B). The antigen in the sample of interest binds to its antibody (FIG. 3C). Next, a known amount of enzyme-secondary antibody is introduced to the area with immobilized antibodies and sample of interest (FIG. 3D). The enzyme-secondary antibody then binds an antigen (FIG. 3E). Next, the free enzyme-secondary antibody is transported to a SERS active area with Raman active molecules immobilized on the SERS substrate (FIG. 3F). Thereafter, the enzyme-secondary antibody reacts with its immobilized Raman active substrate (FIG. 3G). As a result, the Raman active molecule is modified by the enzyme (FIG. 3H). This change is then quantified in time as the Raman spectrum changes (FIG. 3I).

The Raman spectrum changes in time proportionally to the amount of enzyme-secondary antibody present in the SERS active area. In the high concentration case (FIG. 3I, right panel), it takes about 10 minutes for the red spectrum to change to the pink spectrum, where it only takes 5 minutes to achieve the same change in the low concentration case (FIG. 3I, left panel, blue line). After 10 minutes, the spectrum of the low concentration case (FIG. 3I, left panel, pink line) has considerably changed compared to the spectrum at 0 minutes (FIG. 3I, left panel, red line) and the spectrum of the high concentration case at 10 minutes (FIG. 3I, right panel, pink line).

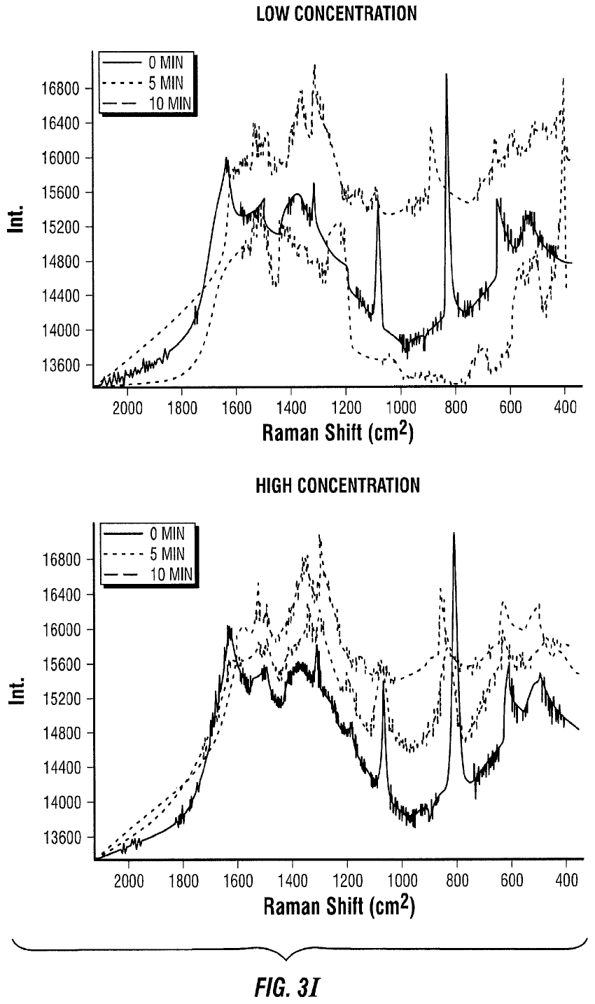

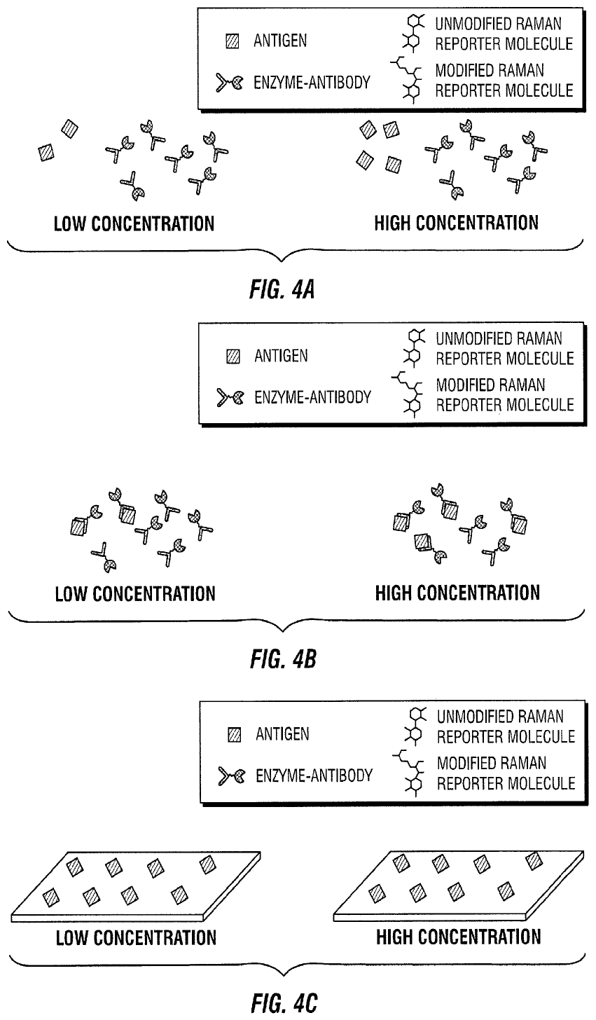

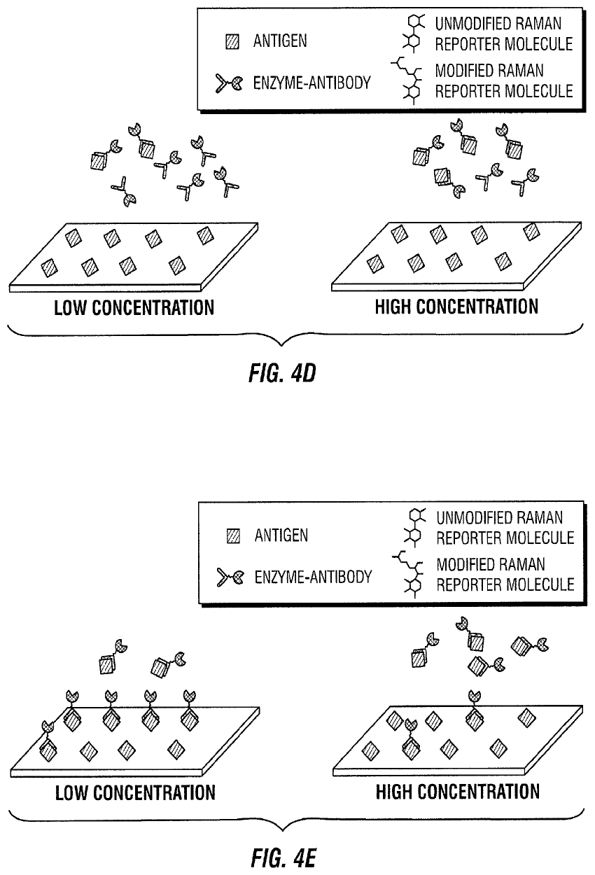

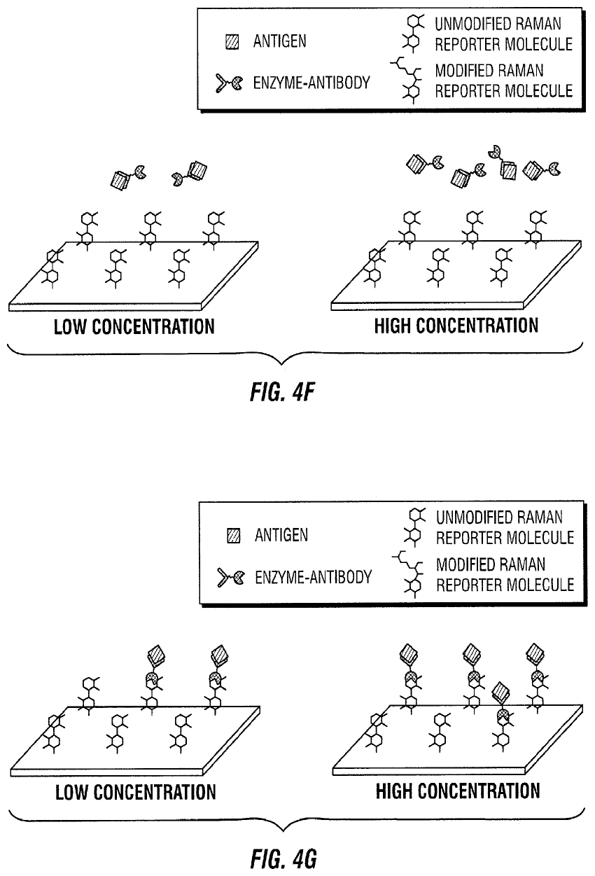

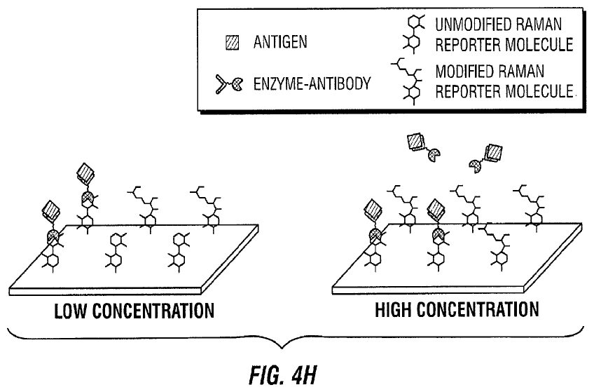

FIG. 4 illustrates a direct assay embodiment. In this embodiment, the sample to be measured is added to a known amount of enzyme-antibody (FIG. 4A). The antigen in the sample of interest binds to its antibody (FIG. 4B). Antigens are also immobilized in a solid support (FIG. C). The sample of interest and the enzyme-antibody are then introduced to the area with immobilized antigens (FIG. 4D). The enzyme-antibody with free binding sites binds to an immobilized antigen (FIG. 4E). Next, the free enzyme-antibody-antigen is transported to a SERS active area with Raman active molecules immobilized on the SERS substrate (FIG. 4F). The enzyme-antibody-antigen reacts with its immobilized Raman active substrate (FIG. 4G). As a result, the Raman active molecule is modified by the enzyme (FIG. 4H). This change is then quantified in time as the Raman spectrum changes (FIG. 4I).

The Raman spectrum changes in time proportionally to the amount of enzyme-antibody-antigen present in the SERS active area. In the low concentration case (FIG. 4I, left panel), it takes 10 minutes for the red spectrum to change to the pink spectrum, where it only takes 5 minutes to achieve the same change in the high concentration case (FIG. 4I, right panel, blue line). After 10 minutes, the spectrum of the high concentration case (FIG. 4I, right panel, pink line) has considerably changed compared to the spectrum at 0 minutes (FIG. 4I, right panel, red line) and the spectrum of the low concentration case at 10 minutes (FIG. 4I, left panel, pink line).

### EXAMPLE 2

### Fabrication and Testing of Enzymatic SERS Sensors

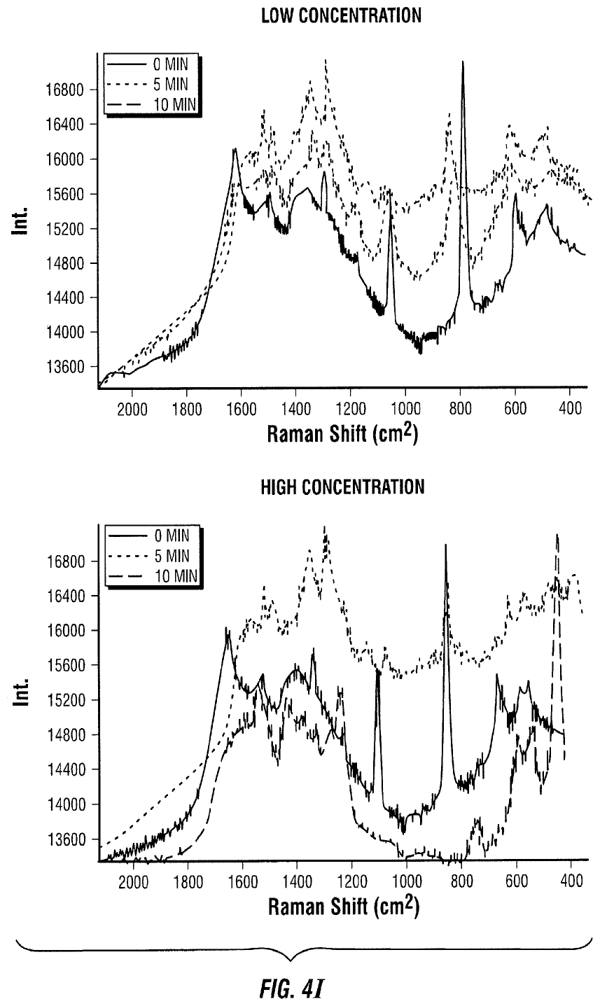

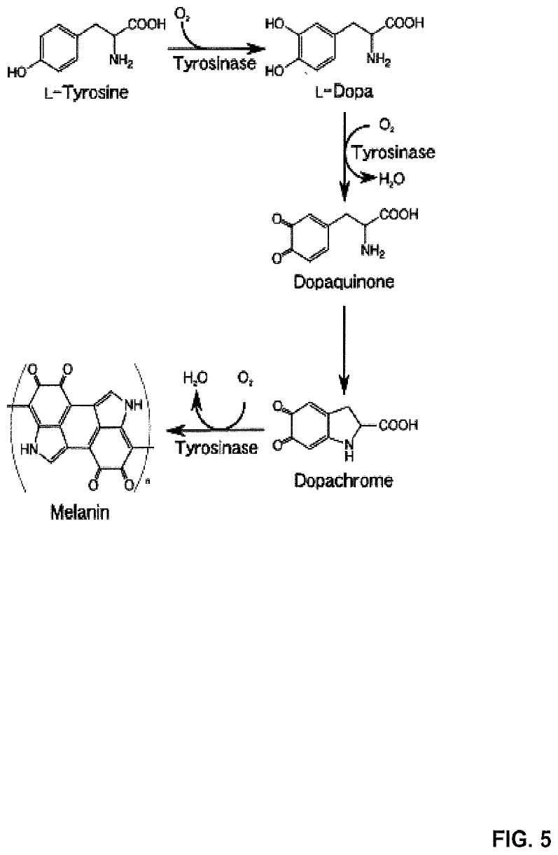

This Example demonstrates a method by which SERS sensors could be fabricated and tested. L-tyrosine was adsorbed on silver nanoparticles. Same amounts of the nanoparticles coated with L-tyrosine were then added to different wells where their SERS spectrum was measured. Thereafter, different amounts of the enzyme Tyrosinase were added to each well. The SERS spectrum of the solution of nanoparticles with the enzyme was measured at different points in time. The SERS spectrum rates of change from each well were compared. The enzymatic reaction is illustrated in FIG. 5.

### EXAMPLE 2.1

### Enzymatic Reaction SERS Response Test Procedure

First, 200 μL of L-tyrosine (34 g/L) was added to 1 mL of silver nanoparticles (1.1×10⁻⁹ M). Next, 0.5 mg of Tyrosinase was added to 1 mL of deionized (DI) water. Thereafter, about 10 mg of bovine serum albumin (BSA) was added to 1 mL of water. Next, about 15 μL of L-tyrosine coated silver nanoparticles were added to 5 wells. Appropriate volumes of DI water were then added to each well according to Table 1.

TABLE 1
Assay procedures.

| | | Well 2 | Well 3 | Well 4 | |
|---|---|---|---|---|---|
| | Well 1 | (C) | (C1/2) | (C1/10) | Well 5 |
| Nanoparticles (μL) | 15 | 15 | 15 | 15 | 15 |
| Water (μL) | 15 | 5 | 10 | 14 | 5 |
| Tyrosinase (μL) | 0 | 10 | 5 | 1 | 0 |
| BSA (μL) | 0 | 0 | 0 | 0 | 10 |

Next, appropriate volumes of Tyrosinase or BSA were added to the wells to be tested according to Table 1. The SERS spectrum was measured approximately two times per minute for 15 minutes for the well. The same process was repeated for the other wells.

### EXAMPLE 2.2

### Evaluation of Results

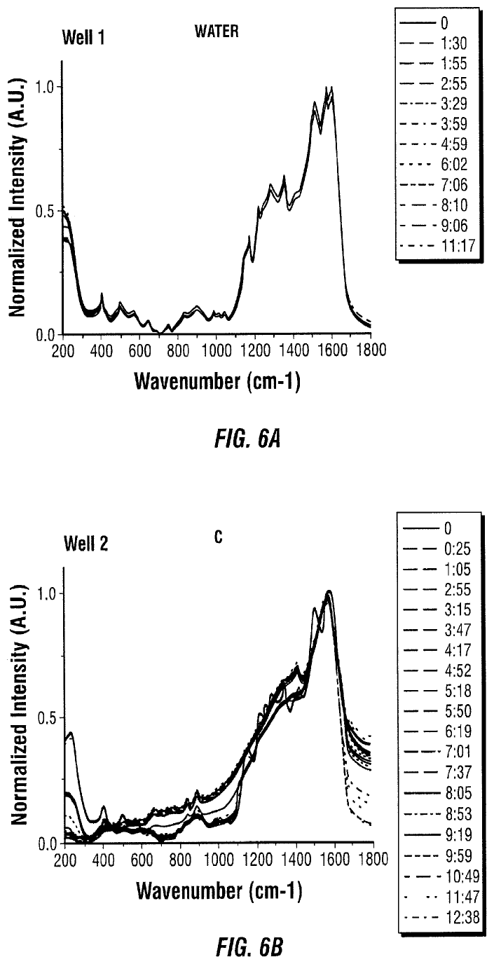

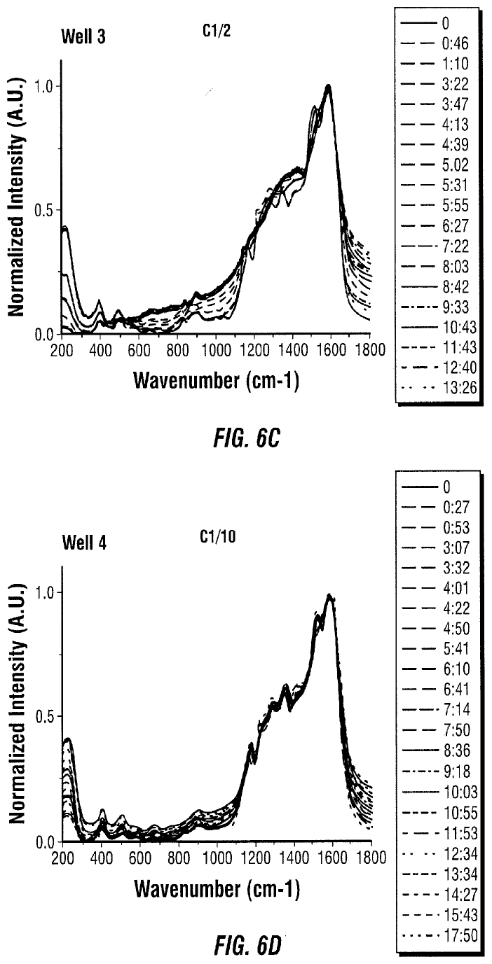

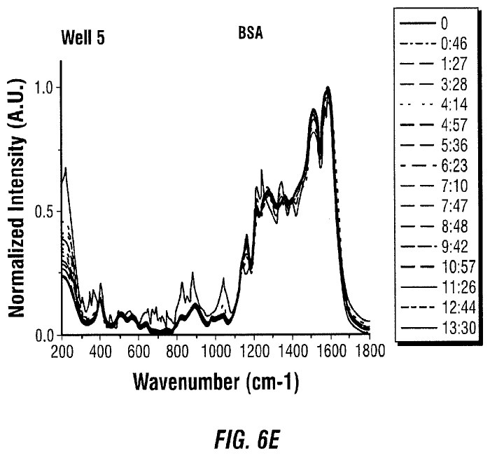

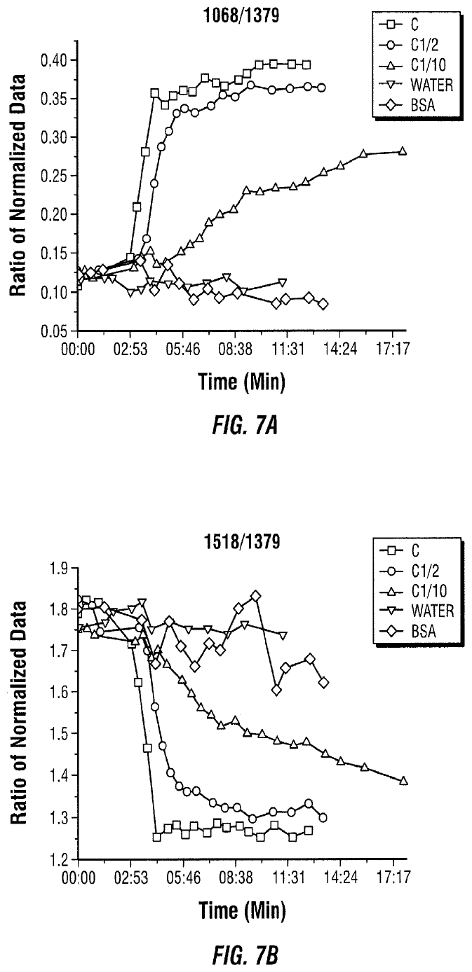

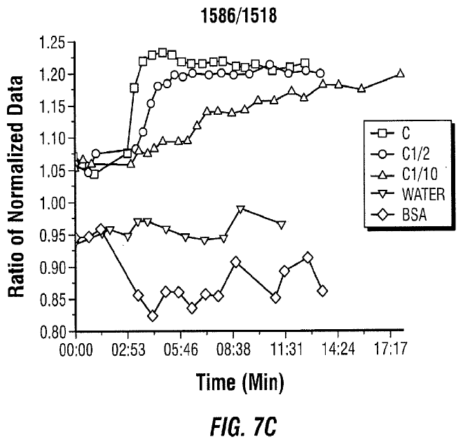

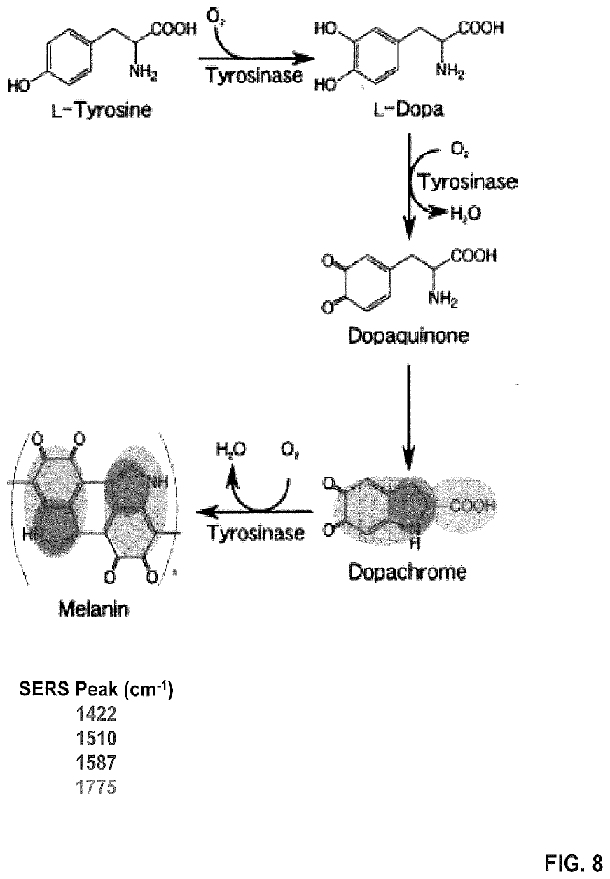

The obtained SERS spectra for each well are shown in FIG. 6. Several wavenumbers were selected to monitor their ratios over time. Such monitored ratios are shown in FIG. 7. The literature was consulted to determine the peak assignments of the changing spectrum. It was found that the peaks obtained in the spectrum after adding tyrosinase indicate that the products of the reaction are formed (FIG. 8 and Table 2).

TABLE 2
Raman peak assignments.

| SERS Peak (cm−1) | Assignment |
|---|---|
| 1075 | C—H in plane deformation |
| 1422 | C═C, C═N in plane vibration in pyrrole |
| 1510 | Indole ring vibration, C═C in plane vibration |
| | in pyrrole |
| 1587 | Indole ring vibration, melanin characteristic |
| | Raman peak, deformation of aromatic ring, |
| | deformation of catechol |
| 1775 | C═O stretching in COOH |

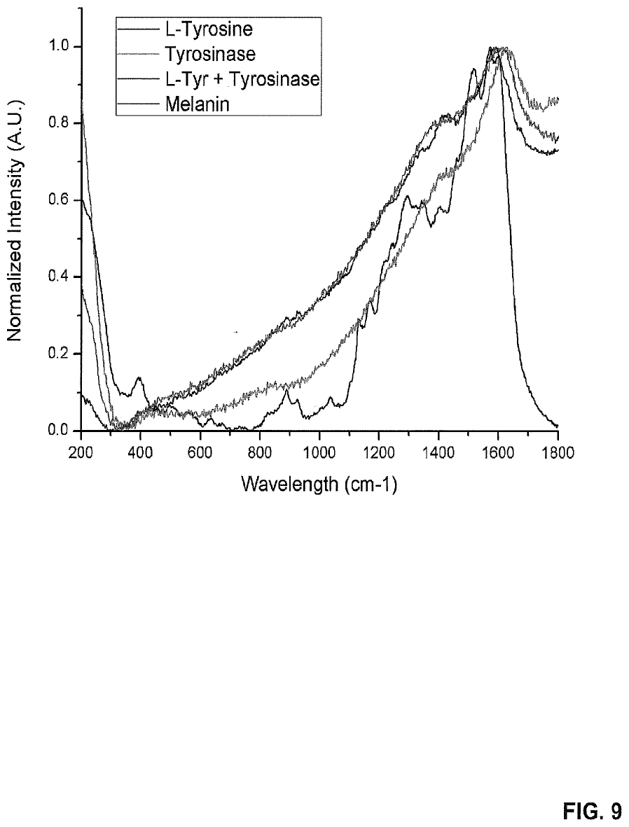

The SERS spectra of L-tyrosine in silver nanoparticles (Ag NPs), tyrosinase in Ag NPs, the product of the reaction between L-tyrosine and tyrosinase in Ag NPs, and melanin in Ag NPs were measured, normalized, and plotted in the same graph to compare them (FIG. 9). The spectrum of the product was almost identical to the one of melanin, indicating that the enzymatic reaction occurred on the surface of the nanoparticles and that the change in spectrum is not due to tyrosinase binding to the nanoparticles.

### EXAMPLE 2.3

### Surface Reaction Test

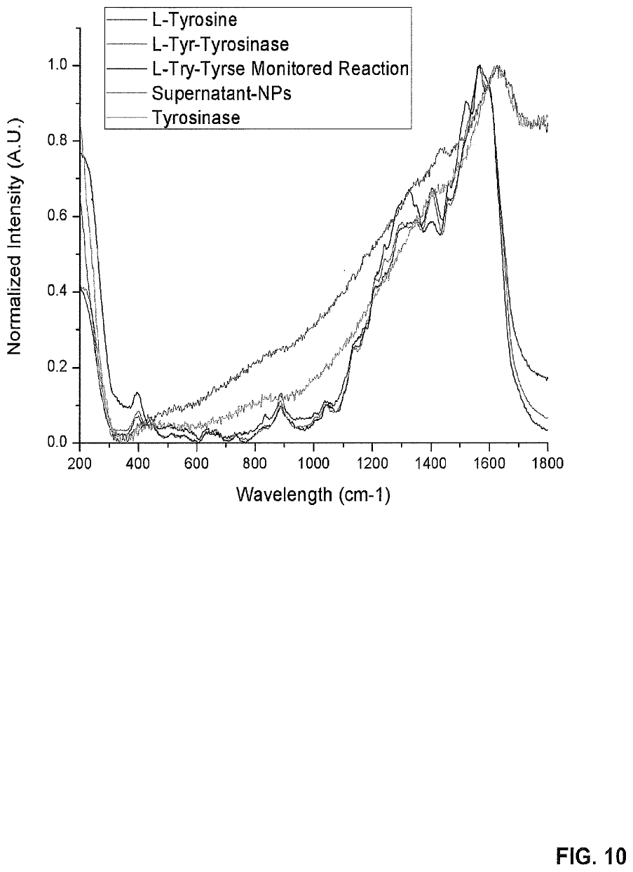

A test was also performed to confirm that the reaction occurs on the surface of the nanoparticles. In this test, tyrosinase was added to L-tyrosine attached to Ag nanoparticles, and the solution was incubated for 20 minutes. Next, the nanoparticles were centrifuged and the supernatant was collected. The nanoparticles were redispersed in water, and the supernatant was added to Ag nanoparticles so that solution components would adsorb on them. The SERS spectra of the centrifuged nanoparticles, the supernatant on Ag nanoparticles, L-tyrosine on nanoparticles, and tyrosinase on nanoparticles were measured. The spectra were normalized and plotted in the same graph (FIG. 10). The product of the L-tyrosine-tyrosinase reaction monitored over time in previous experiments was also included in the plot. The experiment proves that tyrosinase is mostly found in the supernatant, which means that modified L-tyrosine keeps attached to the nanoparticles after the reaction on the surface.

### EXAMPLE 2.4

### Conjugation of Tyrosinase with Cardiac Troponin I

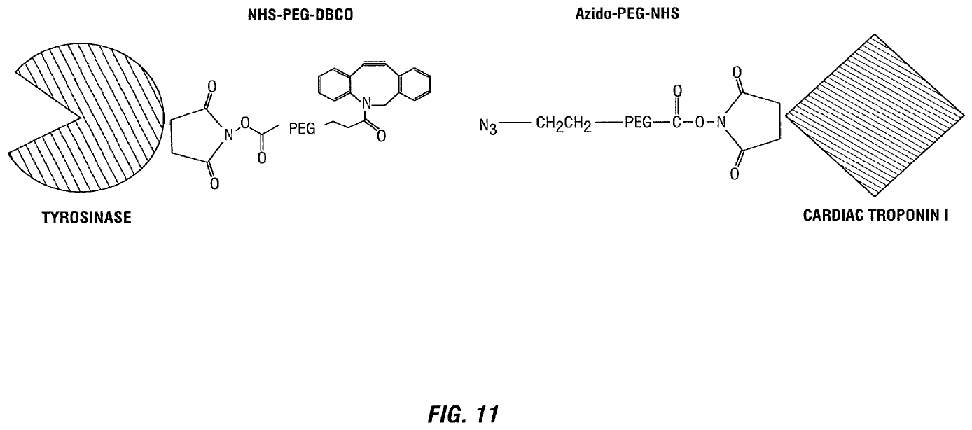

Next, tyrosinase was conjugated with human cardiac troponin I in accordance with the method illustrated in FIG. 11. After conjugation, the response was tested to prove that the enzyme maintains functionality whenever it is conjugated with another protein.

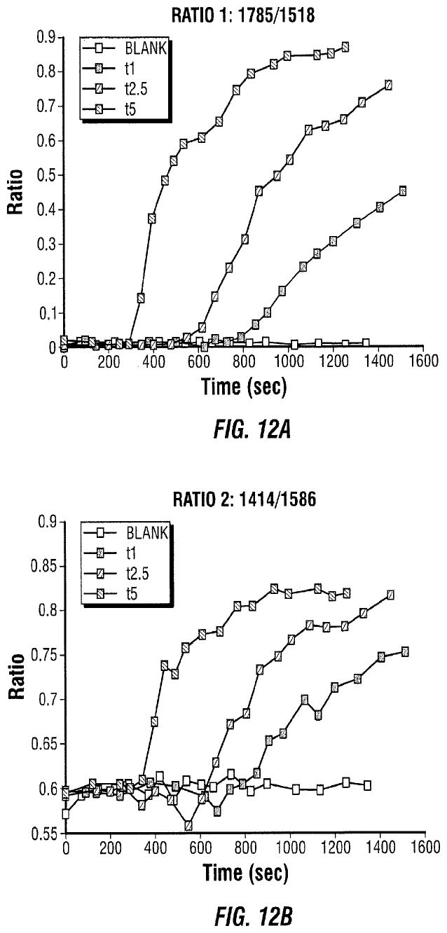

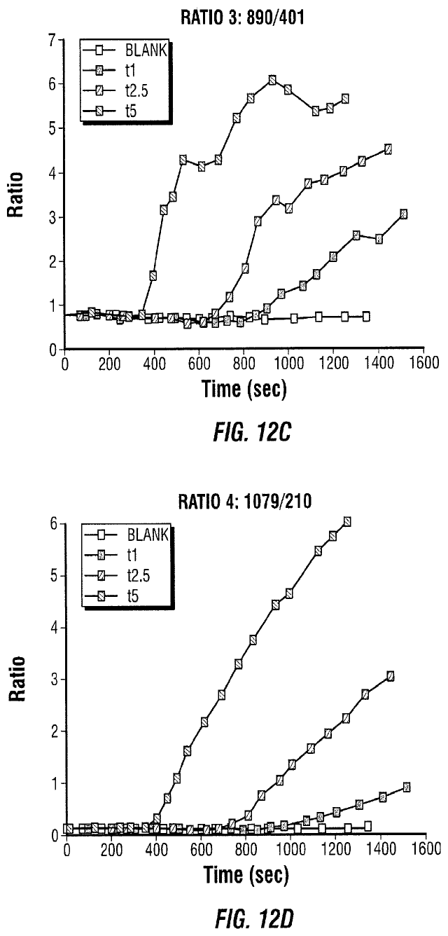

The response observed in FIG. 12 shows that the enzyme-biomolecule conjugation does not affect the enzymatic reaction. This means that it can be used as an assay component. The concentrations t1, t2.5, and t5 mean that 1 μL, 2.5 μL, and 5 μL of tyrosinase was added to 10 μL of L-tyrosine attached to silver nanoparticles and the necessary water to make the final volume 20 μL.

### EXAMPLE 2.5

### Enzyme-Antigen Reaction with Antibody Turbobeads

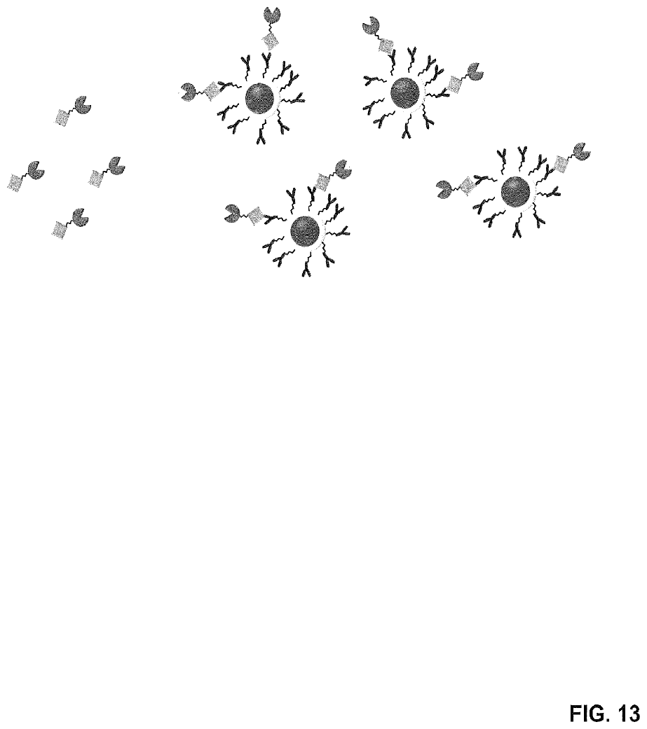

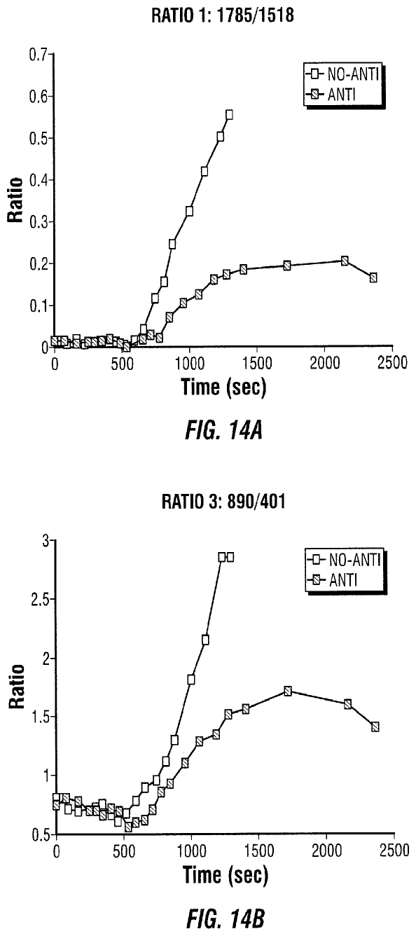

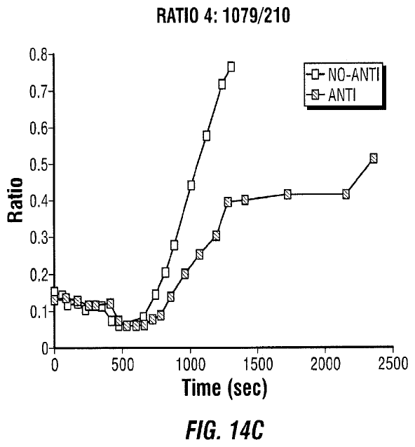

An experiment was performed in which an anti-cardiac troponin I antibody was attached to silica coated magnetic turbobeads. The enzyme-troponin bioconjugate (3 μL) was added to 5 μL of TRIS buffer with and without turbobeads (FIG. 13). When the turbobeads were present, a magnet was used to separate them with the enzyme-troponin bound to them. Then, 5 μL of the supernatant was collected and added to 10 μL of L-tyrosine attached to silver nanoparticles and 5 μL of water. The SERS spectrum was monitored over time as can be observed in FIG. 14. The results indicate that less enzyme-troponin is present in the supernatant when the antibody-turbobeads are present since the SERS spectrum changes at a lower rate. This means that the enzyme-troponin complexes bind to the antibodies. In the case where no antibody-turbobeads were added, the SERS spectrum ratio changed at a faster rate, which means that more complexes are in the supernatant.

### EXAMPLE 2.6

### Reproducibility Tests

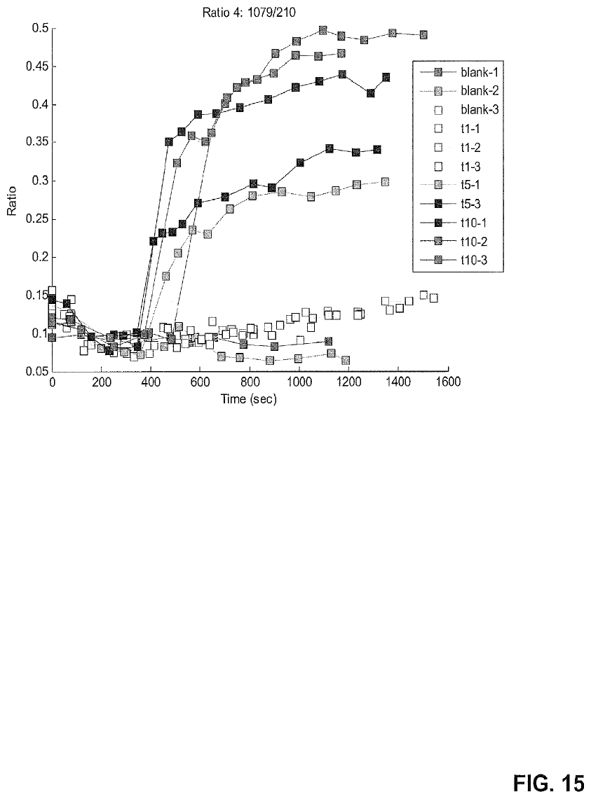

A test was performed in which four measurements (blank, adding 1 μL, 5 μL, and 10 μL of tyrosinase) were repeated three times to test the reproducibility (one measurement, t5, was eliminated in one sample because of instrument errors). The results indicate a level of reproducibility (FIG. 15).

### EXAMPLE 3

### Additional Enzymatic Assays

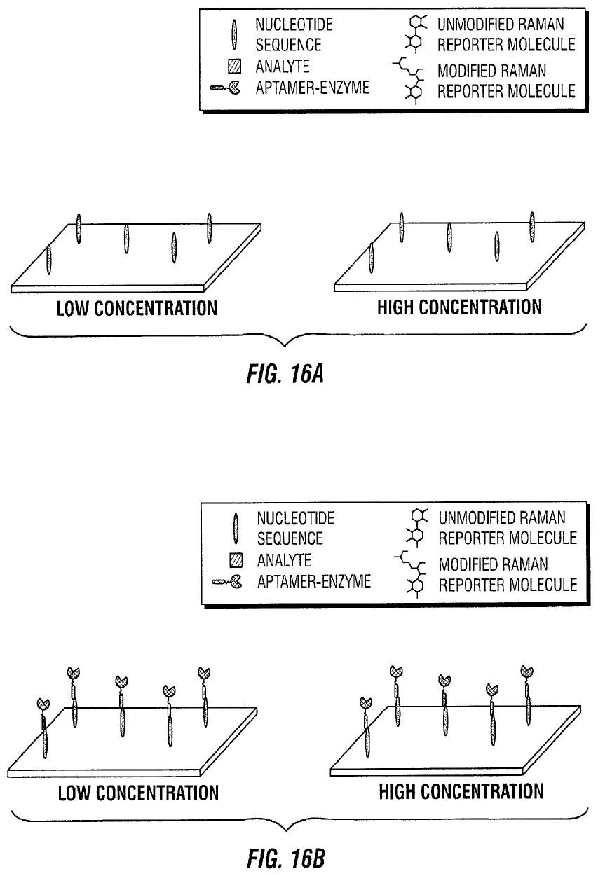

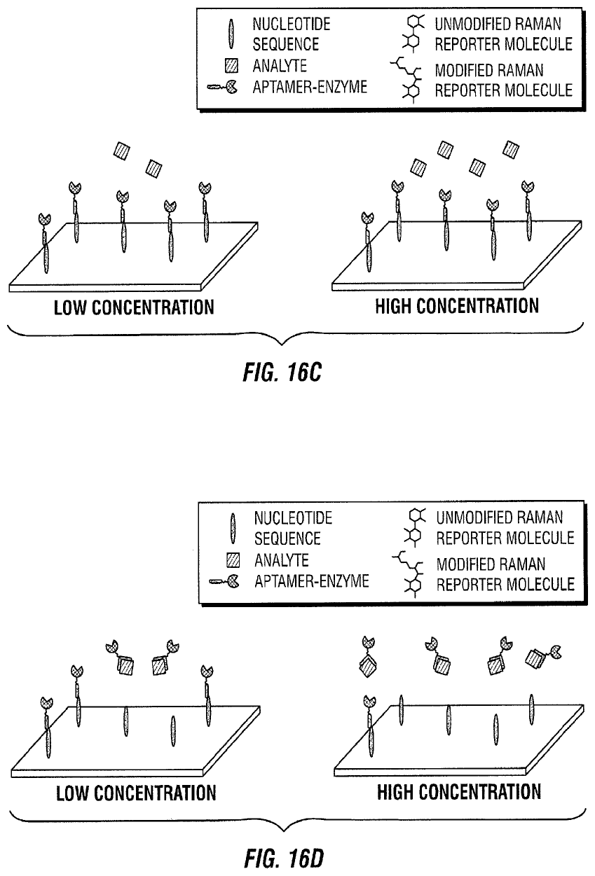

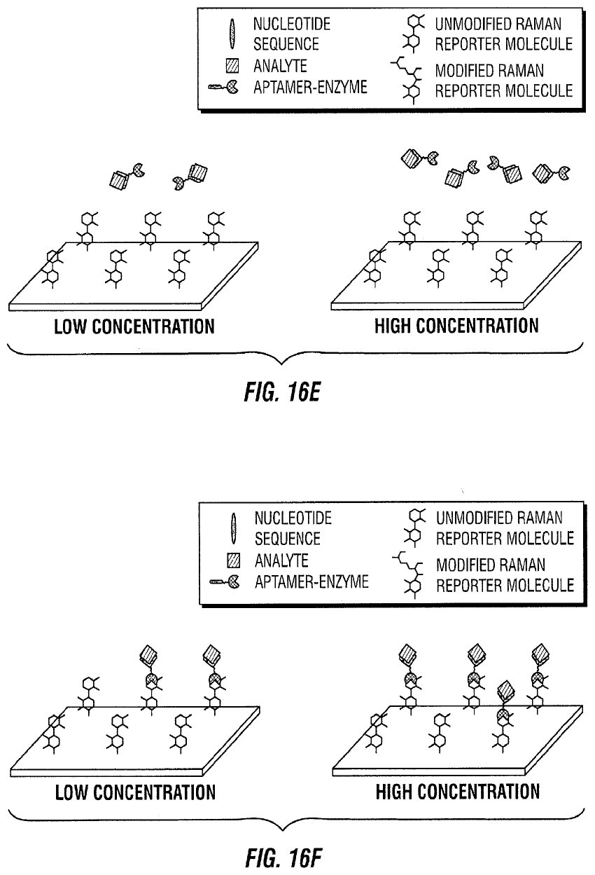

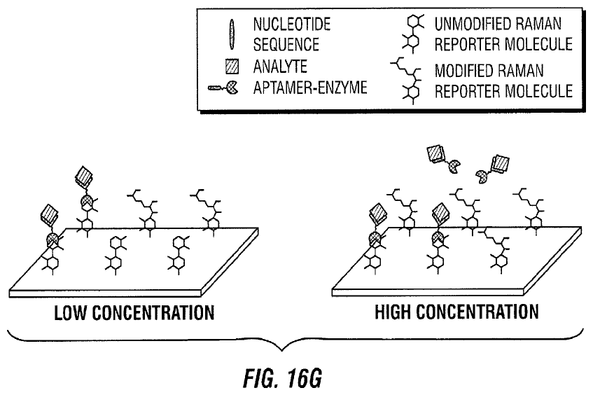

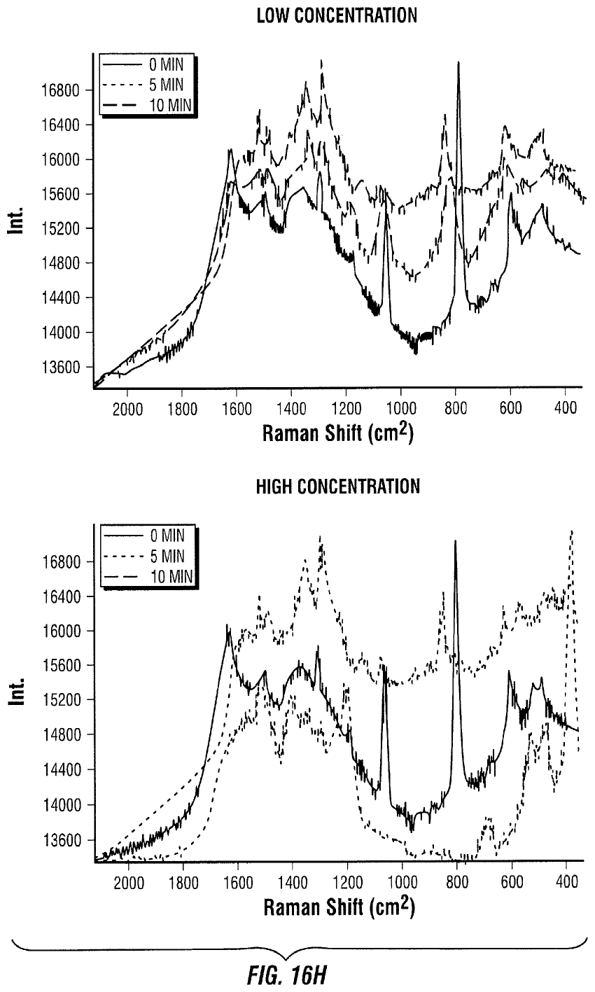

This Example provides three additional embodiments of enzymatic assays. In two of these embodiments, the enzyme used is not linked to a molecule. In addition, in these two embodiments an inhibitor is used. However, the measurement steps resemble the measurement steps in Examples 1-2. The aforementioned embodiments are illustrated in FIGS. 16-18.

FIG. 16 illustrates an enzyme-linked molecule release assay embodiment. In this embodiment, an aptamer is conjugated with the enzyme to form an enzyme linked binding agent (aptamer-enzyme). A nucleotide sequence that binds to part of the aptamer is immobilized on a solid support (FIG. 16A). Next, the aptamer-enzyme is introduced to the solid support such that the aptamer can bind to the immobilized nucleotide sequence (FIG. 16B). As a result, the aptamer-enzyme is immobilized on the solid support through the binding of the nucleotide sequence and the aptamer (FIG. 16B). Next, the analyte that will be measured (i.e., analyte of interest) is introduced to the area with the bound aptamer-enzyme (FIG. 16C). As a result, the aptamer-enzyme binds to the analyte of interest and is released from the solid surface (FIG. 16D). The amount of aptamer-enzyme that is released is dependent on the amount of analyte of interest present.

The free aptamer-enzyme-analyte is then transported to a SERS active area with Raman active molecules immobilized on the SERS substrate (FIG. 16E). The Raman active molecules may be linked or adsorbed to the SERS active area, which is a metallic surface that enhances its Raman signal to produce SERS.

Thereafter, the aptamer-enzyme-analyte reacts with its immobilized Raman active substrate (FIG. 16F). As a result, the Raman active molecule is modified by the enzyme (FIG. 16G). For instance, the enzyme interacts with the Raman active molecule to change its chemical composition.

The Raman spectrum changes in time proportionally to the amount of aptamer-enzyme-analyte present in the SERS active area (FIG. 16H). In particular, the change rate is proportional to the amount of aptamer-enzyme present, which in turn is proportional to the amount of analyte of interest introduced. Therefore, with a calibration curve, this method can be used to quantify the analyte of interest.

In the low concentration case illustrated in the left panel of FIG. 16, it takes 10 minutes for the red spectrum to change to the pink spectrum. However, in the high concentration case illustrated in the right panel of FIG. 16, it only takes 5 minutes to achieve the same change. After 10 minutes, the spectrum of the high concentration case (pink) has considerably changed compared to the spectrum at 0 minutes (red) and the spectrum of the low concentration case at 10 minutes (pink).

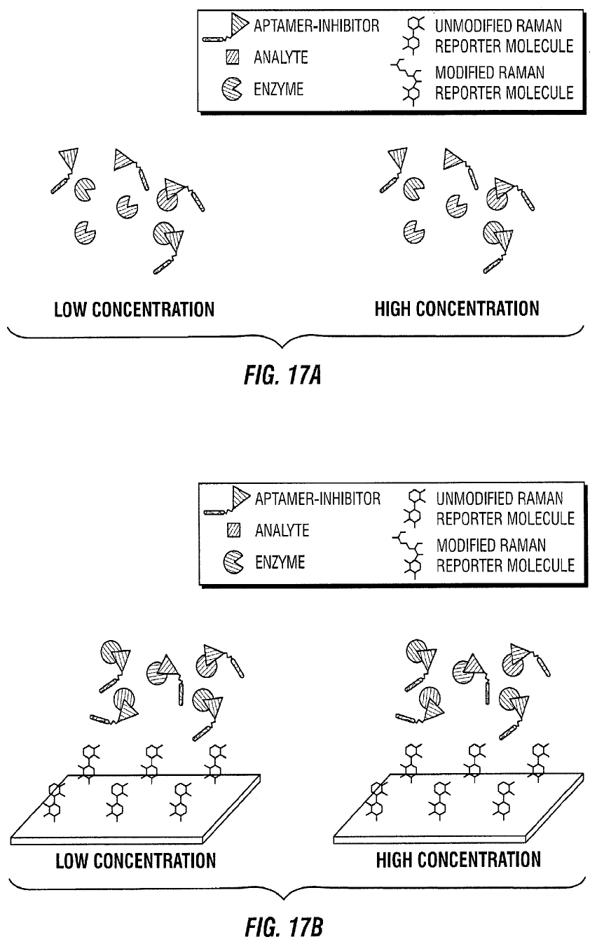

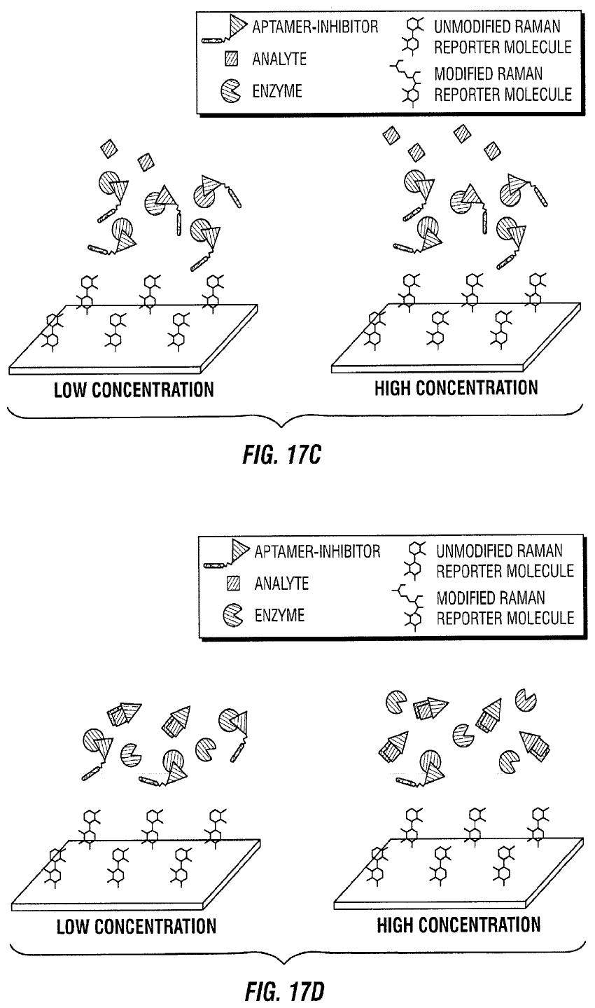

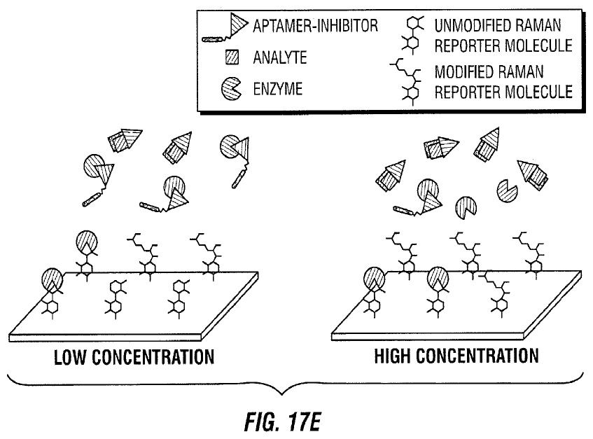

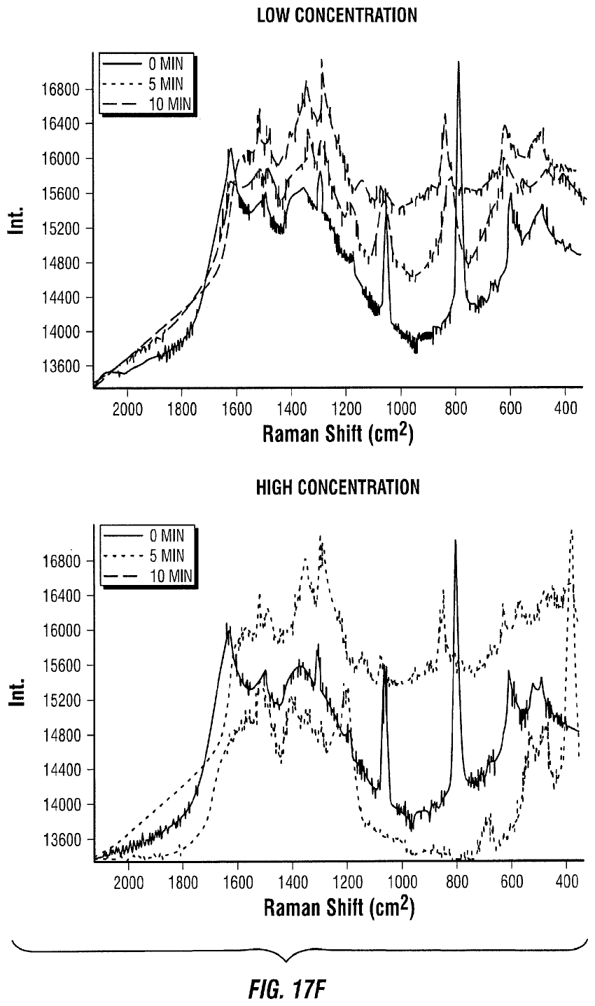

FIG. 17 illustrates a directly proportional inhibiting assay embodiment. In this embodiment, an active aptamer-inhibitor is mixed with the enzyme. The aptamer-inhibitor binds with the enzyme to inhibit its activity and form an aptamer-inhibitor-enzyme complex (FIG. 17A). The aptamer-inhibitor molecules that do not bind to the enzyme are washed away. The aptamer-inhibitor-enzyme complex is then introduced to the SERS active area with Raman active molecules immobilized on the SERS substrate (FIG. 17B). The analyte of interest is then introduced (FIG. 17C). The aptamer-inhibitor binds with the analyte, and this binding prevents the inhibitor from inhibiting the enzyme (FIG. 17D). When this happens, the enzyme is not inhibited and can react with the Raman active molecule. As a result, the Raman active molecule is modified by the active enzyme and this change is quantified in time as the Raman spectrum changes (FIG. 17E). The Raman spectrum changes in time proportionally to the amount of active enzyme (i.e., enzyme that is not inhibited by the inhibitor) present in the SERS active area, which in turn is proportional to the amount of analyte of interest introduced as this affects how much inhibitor is active (FIG. 17F). Therefore, with a calibration curve, this method can also be used to quantify the analyte of interest.

In the low concentration case illustrated on the left panel of FIG. 17, it takes 10 minutes for the red spectrum to change to the pink spectrum. However, in the high concentration case illustrated on the right panel of FIG. 17, it only takes 5 minutes to achieve the same change. After 10 minutes, the spectrum of the high concentration case (pink) has considerably changed compared to the spectrum at 0 minutes (red) and the spectrum of the low concentration case at 10 minutes (pink).

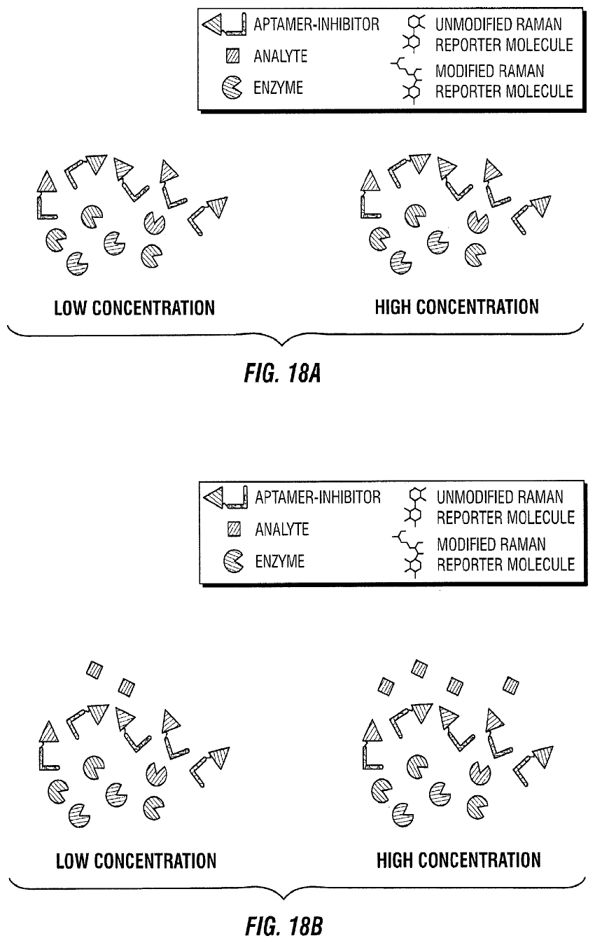

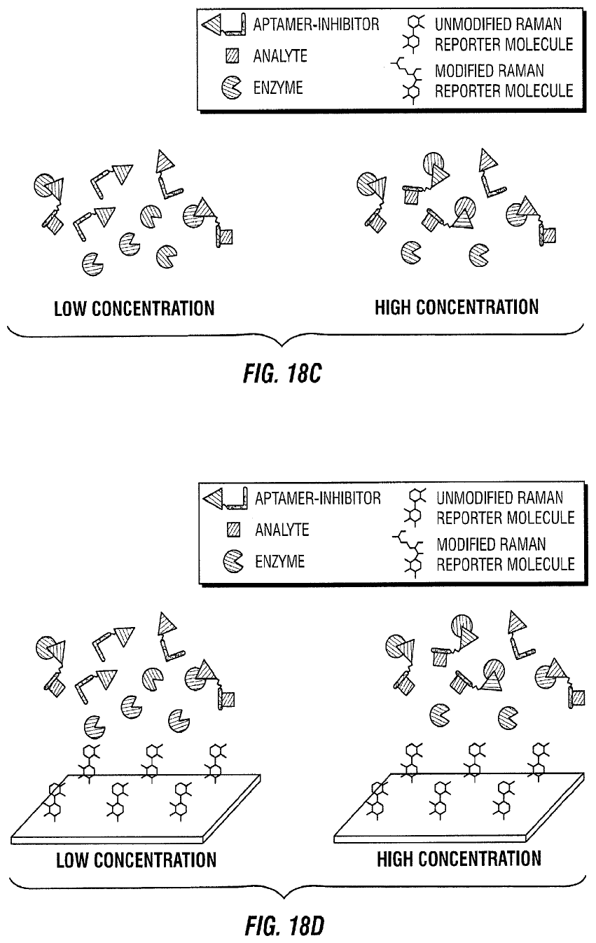

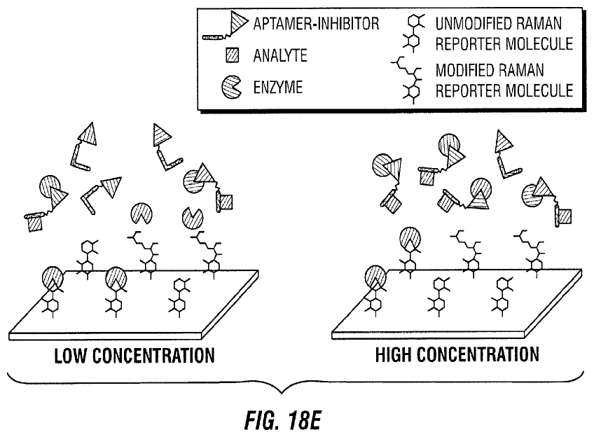

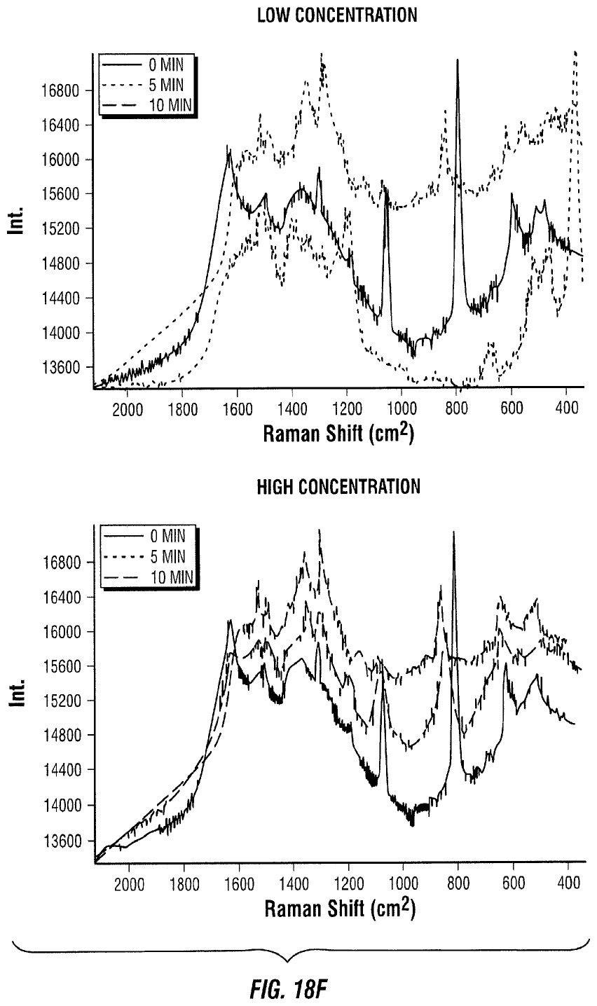

FIG. 18 illustrates an inversely proportional inhibiting assay embodiment. In this embodiment, an inhibitor of the enzyme or a molecule that prevents the enzyme from reacting with its substrate is used. This inhibitor is conjugated with an aptamer to form an inactive aptamer-inhibitor complex. The inactive aptamer-inhibitor is first mixed with the enzyme (FIG. 18A). When the aptamer is not bound with its analyte, the inhibitor is not active or it does not inhibit the enzyme. Therefore, the enzyme is active. The analyte of interest is then introduced (FIG. 18B). The aptamer-inhibitor binds with the analyte, and this binding activates the inhibitor to inhibit the enzyme (FIG. 18C). Therefore, the amount of enzyme that is inhibited is dependent on the amount of analyte of interest present.

Next, the assay components are transported to a SERS active area with Raman active molecules immobilized on the SERS substrate (FIG. 18D). The Raman active molecule is modified by the active enzyme and this change is quantified in time as the Raman spectrum changes (FIG. 18E). The Raman spectrum changes in time proportionally to the amount of active enzyme present in the SERS active area (FIG. 18F), which in turn is proportional to the amount of analyte of interest introduced as this affects how much inhibitor is active. Therefore, with a calibration curve, this method can be used to quantify the analyte of interest.

In the high concentration case illustrated on the right panel of FIG. 18, it takes 10 minutes for the red spectrum to change to the pink spectrum. However, in the low concentration case illustrated on the left panel of FIG. 18, it only takes 5 minutes to achieve the same change. After 10 minutes, the spectrum of the low concentration case (pink) has considerably changed compared to the spectrum at 0 min (red) and the spectrum of the high concentration case at 10 min (pink).

In the embodiments illustrated in FIGS. 17-18, an aptamer is conjugated with an inhibitor to form the aptamer-inhibitor complex. An aptamer was mentioned because it is an exemplary molecular structure that can change shape when it binds to its target. This change in shape is what affects the inhibitor and determines if the inhibitor is active or not. However, in additional embodiments, the binding of the analyte of interest to other agents (e.g., aptamers, antibodies, DNA, etc.) can also cause a change that will affect the activity of the enzyme or the activity of the enzyme inhibitor.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of quantifying an analyte concentration in a sample, said method comprising:
providing a pre-screened amount of an enzyme, wherein the enzyme is pre-screened against the analyte in the sample;
associating the pre-screened enzyme with a Raman reporter molecule, wherein the Raman reporter molecule reacts with the pre-screened enzyme, and wherein the reaction results in a change in the Raman spectrum pattern of the Raman reporter molecule;
detecting the change in the Raman spectrum pattern of the Raman reporter molecule; and
correlating the change in the Raman spectrum pattern of the Raman reporter molecule to the concentration of the analyte in the sample.

2. The method of claim 1, wherein the pre-screening of the enzyme against the analyte in the sample affects the concentration of the enzyme in proportion to the amount of the analyte in the sample.

3. The method of claim 2, wherein the pre-screening of the enzyme against the analyte in the sample increases the concentration of the enzyme in proportion to the amount of the analyte in the sample.

4. The method of claim 2, wherein the pre-screening of the enzyme against the analyte in the sample reduces the concentration of the enzyme in proportion to the amount of the analyte in the sample.

5. The method of claim 1, wherein the pre-screening of the enzyme against the analyte in the sample reduces the enzymatic activity of the enzyme in proportion to the amount of the analyte in the sample.

6. The method of claim 1, wherein the pre-screening of the enzyme against the analyte in the sample increases the enzymatic activity of the enzyme in proportion to the amount of the analyte in the sample.

7. The method of claim 1, wherein the enzyme is an enzyme-linked analyte.

8. The method of claim 7, wherein the enzyme-linked analyte is pre-screened against the analyte by the following steps:
associating the analyte from the sample with an immobilized binding agent specific for the analyte, wherein the associating results in the binding of at least some of the analytes to the immobilized binding agent;
adding a known amount of the enzyme-linked analyte to the immobilized binding agent, wherein the adding results in the binding of at least some of the enzyme-linked analyte to the immobilized binding agent; and
separating the unbound enzyme-linked analyte to provide the pre-screened amount of the enzyme.

9. The method of claim 1, wherein the enzyme is an enzyme-linked analyte binding agent.

10. The method of claim 9, wherein the enzyme-linked analyte binding agent is pre-screened against the analyte by the following steps:
associating the analyte from the sample with an immobilized binding agent specific for the analyte, wherein the associating results in the binding of at least some of the analytes to the immobilized binding agent;
adding a known amount of the enzyme-linked analyte binding agent to the immobilized binding agent, wherein the adding results in the binding of at least some of the enzyme-linked analyte binding agent to the analyte bound to the immobilized binding agent; and
separating the unbound enzyme-linked analyte binding agent to provide the pre-screened amount of the enzyme.

11. The method of claim 9, wherein the enzyme-linked analyte binding agent is pre-screened against the analyte by the following steps:
mixing a known amount of the enzyme-linked analyte binding agent with the analyte from the sample, wherein the mixing results in the binding of at least some of the enzyme-linked analyte binding agents to the analyte to form enzyme-linked analyte binding agent-analyte complexes;
associating the enzyme-linked analyte binding agent-analyte complexes with an immobilized binding agent specific for the analyte, wherein the associating results in the binding of at least some of the enzyme-linked analyte binding agent-analyte complexes to the immobilized binding agent; and
separating the unbound enzyme-linked analyte binding agents to provide the pre-screened amount of the enzyme.

12. The method of claim 9, wherein the enzyme-linked analyte binding agent is pre-screened by the following steps:
associating the analyte from the sample with a known amount of the enzyme-linked analyte binding agent, wherein the associating results in the binding of at least some of the analytes to the enzyme-linked analyte binding agent;
adding the enzyme-linked analyte binding agent to immobilized analytes, wherein the adding results in the binding of at least some of the enzyme-linked analyte binding agents to the immobilized analytes; and
separating the enzyme-linked analyte binding agents that are not bound to the immobilized analytes to provide the pre-screened amount of the enzyme.

13. The method of claim 9, wherein the enzyme-linked analyte binding agent is pre-screened against the analyte by the following steps:
associating a known amount of the enzyme-linked analyte binding agent with an immobilized binding agent specific for the enzyme-linked analyte binding agent, wherein the associating results in the binding of at least some of the enzyme-linked analyte binding agents to the immobilized binding agents;
adding the analyte from the sample to the immobilized binding agents, wherein the adding results in the binding of at least some of the enzyme-linked analyte binding agents to the analyte to form enzyme-linked analyte binding agent-analyte complexes, and wherein the binding facilitates the release of the enzyme-linked analyte binding agent-analyte complexes from the immobilized surface; and
separating the released enzyme-linked analyte binding agent-analyte complexes to provide the pre-screened amount of the enzyme.

14. The method of claim 1, wherein the enzyme is pre-screened by the following steps:
associating a known amount of the enzyme with an enzyme inhibitor comprising an analyte binding agent,
wherein the associating results in the binding of at least some of the enzyme inhibitors to at least some of the enzymes to form enzyme-inhibitor complexes; and
adding the analyte from the sample to the enzyme-inhibitor complexes,
wherein the adding results in the binding of at least some of the analytes to the analyte binding agent of the enzyme-inhibitor complexes, and
wherein the binding counteracts the inhibition of at least some of the enzymes by the enzyme inhibitors in the sample, and
wherein the enzymes that are not inhibited by the enzyme inhibitors provide the pre-screened amount of the enzyme.

15. The method of claim 14, wherein the binding counteracts the inhibition of at least some of the enzymes by the enzyme inhibitors by facilitating the release of at least some of the enzyme inhibitors from at least some of the enzyme-inhibitor complexes

16. The method of claim 14, wherein the binding counteracts the inhibition of at least some of the enzymes by the enzyme inhibitors by inactivating or modifying the enzyme inhibition activity of the enzyme inhibitors.

17. The method of claim 14, wherein the binding counteracts the inhibition of at least some of the enzymes by the enzyme inhibitors by modifying the structure of the enzyme inhibitors.

18. The method of claim 1, wherein the enzyme is pre-screened by the following steps:
associating the analyte from the sample with a known amount of the enzyme and an enzyme inhibitor comprising an analyte binding agent,
wherein the associating results in the binding of at least some of the analytes to the analyte binding agent of the enzyme inhibitor,
wherein the binding facilitates the inhibition of at least some of the enzymes by the enzyme inhibitors in the sample, and
wherein the enzymes that are not inhibited provide the pre-screened amount of the enzyme.

19. The method of claim 18, wherein the binding facilitates the inhibition of at least some of the enzymes by the enzyme inhibitor by facilitating the binding of the enzyme inhibitor to at least some of the enzymes.

20. The method of claim 18, wherein the binding facilitates the inhibition of at least some of the enzymes by the enzyme inhibitor by activating the enzyme inhibitor.

21. The method of claim 18, wherein the activation occurs by modifying the structure of the enzyme inhibitor.

22. The method of claim 1, wherein the analyte is selected from the group consisting of antigens, proteins, peptides, small molecules, DNA strands, oligonucleotides, and combinations thereof.

23. The method of claim 1, wherein the enzyme comprises tyrosinase.

24. The method of claim 1, wherein the Raman reporter molecule is L-tyrosine.

25. The method of claim 1, wherein the Raman reporter molecule is immobilized on a surface.

26. The method of claim 25, wherein the surface is a surface enhanced Raman spectroscopy (SERS) active area.

27. The method of claim 1, wherein the reaction of the Raman reporter molecule with the enzyme results in a structural change in the Raman reporter molecule.

28. The method of claim 1, wherein the change in the Raman spectrum pattern of the Raman reporter molecule is detected over time.

29. The method of claim 28, wherein the change in the Raman spectrum pattern of the Raman reporter molecule is detected in real-time.

30. The method of claim 1, wherein the detected change in the Raman spectrum pattern of the Raman reporter molecule is proportional to the amount of the pre-screened enzyme.

31. The method of claim 1, wherein the detected change comprises a detected change rate.

32. The method of claim 1, wherein the correlating comprises:
correlating the change over time in the Raman spectrum pattern of the Raman reporter molecule to the concentration of the pre-screened amount of the enzyme; and
correlating the concentration of the pre-screened amount of the enzyme to the concentration of the analyte in the sample.

33. The method of claim 1, wherein the correlating comprises comparing the change in the Raman spectrum pattern of the Raman reporter molecule to a calibration curve, wherein the calibration curve represents different known amounts of the analyte.
